(19) United States
(12) Patent Application Publication
(10) Pub. No.: US 2026/0182822 A1
(43) Pub. Date: Jul. 2, 2026

(54) INFORMATION PROCESSING DEVICE, ENDOSCOPIC SYSTEM, AND METHOD FOR CONTROLLING INFORMATION PROCESSING DEVICE

(71) Applicant: OLYMPUS MEDICAL SYSTEMS CORP., Tokyo (JP)

(72) Inventors: Fuyuki ONEYAMA, Tokyo (JP); Hirokazu NISHIMURA, Tokyo (JP)

(73) Assignee: OLYMPUS MEDICAL SYSTEMS CORP., Tokyo (JP)

(21) Appl. No.: 19/551,016

(22) Filed: Feb. 26, 2026

Related U.S. Application Data
(63) Continuation of application No. PCT/JP2023/033731, filed on Sep. 15, 2023.

(51) Int. Cl. A61B 1/00; A61B 1/015

(52) U.S. Cl. CPC A61B 1/00006; A61B 1/015

(57) ABSTRACT

An information processing device includes a processor including hardware. The processor acquires a physical attribute of a subject, sets, based on the acquired physical attribute of the subject, a condition related to control information for insertion of an endoscope, and generates, based on the set condition, the control information.

### CROSS REFERENCE TO RELATED APPLICATIONS

This application is a continuation of International Patent Application No. PCT/JP 2023/033731, having an international filing date of Sep. 15, 2023, which designated the United States, the entirety of which is incorporated herein by reference.

### BACKGROUND OF THE INVENTION

There is a demand for technologies to perform endoscopy on regions such as the large intestine without causing a subject's pain. WO 2016/135966 discloses a technique of creating, based on operation data during a past examination and past support information of a subject to which an endoscope is inserted, operation support information including primary information acquired by various sensors attached to the endoscope and secondary information obtained by processing the acquired primary information.

### SUMMARY OF THE INVENTION

In accordance with one of some aspect, there is provided an information processing device comprising a processor including hardware, wherein the processor is configured to: acquire a physical attribute of a subject; set, based on the acquired physical attribute of the subject, a condition related to control information for insertion of an endoscope; and generate, based on the set condition, the control information.

In accordance with one of some aspect, there is provided an endoscopic system comprising: an endoscope configured to be electrically driven; a drive device configured to drive the endoscope; and a processor including hardware, wherein the processor is configured to: acquire a physical attribute of a subject; set, based on the acquired physical attribute of the subject, a condition related to control information for insertion of the endoscope; and generate, based on the set condition, the control information for controlling the drive device.

In accordance with one of some aspect, there is provided a method for controlling an information processing device, wherein the information processing device is configured to: acquire a physical attribute of a subject; set, based on the acquired physical attribute of the subject, a condition related to control information for insertion of an endoscope; and generate, based on the set condition, the control information.

### BRIEF DESCRIPTION OF THE DRAWINGS

FIG. 1 is a diagram illustrating an example configuration of an information processing device according to the present embodiment;

FIG. 2 is a diagram schematically illustrating a large intestine;

FIG. 3 is a diagram illustrating an example configuration of an electric endoscopic system;

FIG. 4 is a diagram illustrating an example configuration of a drive control device;

FIG. 5 is a diagram illustrating an example of a bending portion and a mechanism to drive the bending portion;

FIG. 6 is a diagram illustrating an example configuration of a forward/backward drive device;

FIG. 7 is a diagram illustrating an example configuration of a joint portion including a roll drive device;

FIG. 8 is a diagram illustrating another example configuration of the endoscopic system;

FIG. 9 is a flowchart illustrating example processing for automatic insertion control;

FIG. 10 is a flowchart illustrating example processing for a technique according to the present embodiment;

FIG. 11 is a flowchart illustrating example processing for condition setting;

FIG. 12 is a diagram illustrating a relationship between operational difficulty and control information;

FIG. 13A illustrates a relationship between the age of a subject and a difficulty rank;

FIG. 13B is a diagram illustrating a relationship between the gender of the subject and the difficulty rank;

FIG. 14A is a diagram illustrating a relationship between the BMI of the subject and the difficulty rank;

FIG. 14B is a diagram illustrating a relationship between the height of the subject and the difficulty rank;

FIG. 15 is a diagram illustrating a relationship between combinations of the age and gender of the subject and the difficulty rank;

FIG. 16 is a flowchart illustrating another example processing related to the technique according to the present embodiment;

FIG. 17 is a flowchart illustrating another example processing for the condition setting;

FIG. 18A illustrates a relationship between combinations of a drug to be administrated and the gender of the subject and the difficulty rank;

FIG. 18B is a diagram illustrating a relationship between combinations of past examination information and the gender of the subject and the difficulty rank;

FIG. 19 is a flowchart illustrating example processing for condition change;

FIG. 20 is a diagram illustrating a relationship between the operational difficulty and a manipulation algorithm; and

FIG. 21 is a diagram illustrating an example of information regarding operational difficulty.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

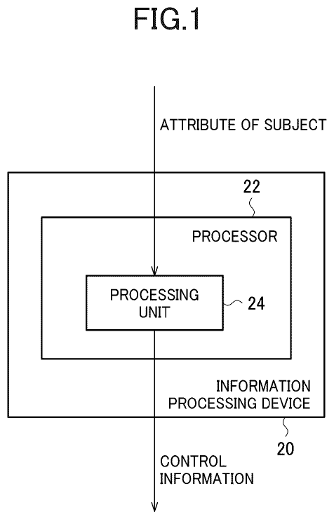

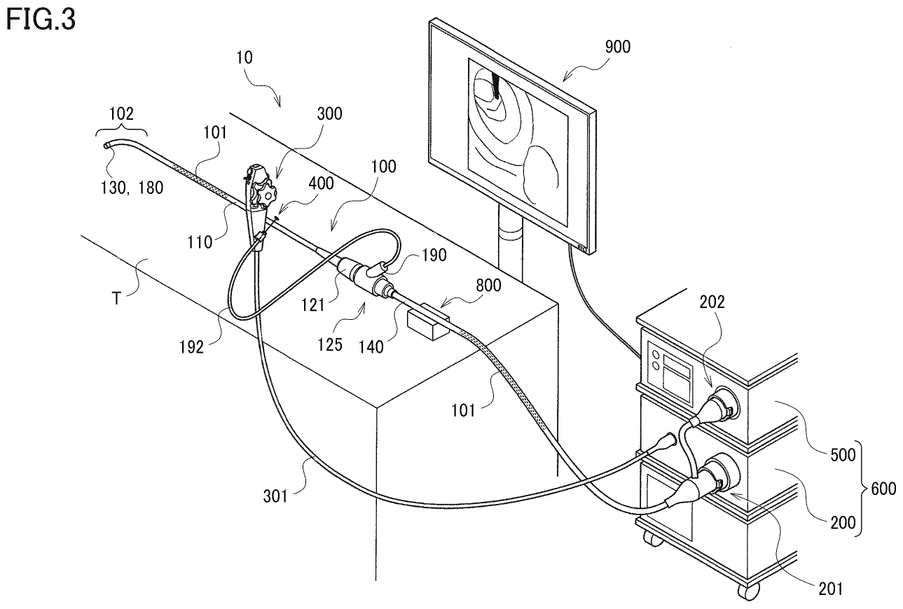

FIG. 1 is a block diagram illustrating an information processing device 20 according to the present embodiment. The information processing device 20 according to the present embodiment includes a processor 22. The processor 22 acquires an attribute of a subject to be described later. The subject means a person who undergoes endoscopy that is an examination using an endoscope 100 to be described later. That is, the technique according to the present embodiment is in relation to the control of the endoscope 100 used for endoscopy. The attribute of the subject means a property or a characteristic that a plurality of subjects have in common and is used for distinguishing one subject from another subject. Specific examples of the attribute are age, gender, height, weight, and BMI, but may be other properties or characteristics, and will be described in detail later. Further, based on the acquired attribute of the subject, the processor 22 generates control information to be described later. The control information means parameters for controlling a drive device that drives the endoscope 100. More specifically, the control information means parameters for controlling a drive control device 200 and the like to be described later in FIG. 3 and the like. That is, the technique according to the present embodiment can also be implemented as an endoscopic system 10 including the information processing device 20 and the endoscope 100. A more detailed specific example of the endoscopic system 10 will be described later with reference to FIG. 3 and subsequent drawings.

The processor 22 according to the present embodiment is constituted by the following hardware. The hardware can include at least one of a circuit that processes digital signals and a circuit that processes analog signals. For example, the hardware can be constituted by one or more circuit devices or one or more circuit elements mounted on a circuit board. The one or more circuit devices are, for example, ICs and the like. The one or more circuit elements are, for example, resistors, capacitors, and the like.

The information processing device 20 according to the present embodiment may also include a memory (not shown) and the processor 22 that operates based on information stored in the memory. With this configuration, the processor 22 can function as a processing unit 24. The information is, for example, programs and various types of data. As the processor 22, a central processing unit (CPU), a graphics processing unit (GPU), a digital signal processor (DSP), or the like can be used. The memory may be a semiconductor memory such as a static random access memory (SRAM) and a dynamic random access memory (DRAM), a register, a magnetic storage device such as a hard disk drive, or an optical storage device such as an optical disk drive. For example, the memory stores instructions readable by a computer. When the instructions are executed by the processor 22, the functions of each part of the processing unit 24 are realized as processing. The instructions here may be instructions as an instruction set constituting a program, or may be instructions that directs the hardware circuit of the processor 22 to operate. The memory is also referred to as storage device. For convenience of description, the entity that performs processing related to the technique according to the present embodiment is uniformly described as the processor 22 unless otherwise specified. However, the processor 22 can be appropriately construed as the processing unit 24 or the like as software.

The above-described program can be stored in, for example, a non-transitory information storage medium, which is a computer-readable medium. The information storage medium can be implemented, for example, as an optical disc, a memory card, an HDD, or a semiconductor memory. The semiconductor memory is, for example, a ROM or a non-volatile memory.

In the subsequent description, large intestine endoscopy is exemplified as the endoscopy related to the technique according to the present embodiment. That is, the endoscope 100 according to the present embodiment is exemplified as a flexible endoscope for large intestine observation. The same applies to a distal end portion 130 and the like included in the endoscope 100. However, the subsequent description does not hinder applying the technique according to the present embodiment to endoscopy other than large intestine endoscopy.

The large intestine endoscopy will be described. It is statistically clear that the colorectal cancer ranks higher for both males and females among organs where cancer as a disease is discovered. The curability of colorectal cancer is high if detected at an early stage, but no subjective symptom is found at the early stage. Therefore, regular medical examinations are recommended to detect colorectal cancer early. If the result of a fecal occult blood test, which is one item of the medical examination, is positive, there is a suspicion of colorectal cancer. Accordingly, large intestine endoscopy to examine the large intestine in more detail is performed. Alternatively, in a case where a suspected symptom of colorectal cancer, such as melena, has been found, large intestine endoscopy is similarly performed. The subject of large intestine endoscopy visits a facility such as a hospital with his/her large intestine emptied through previous treatment or the like to have large intestine endoscopy.

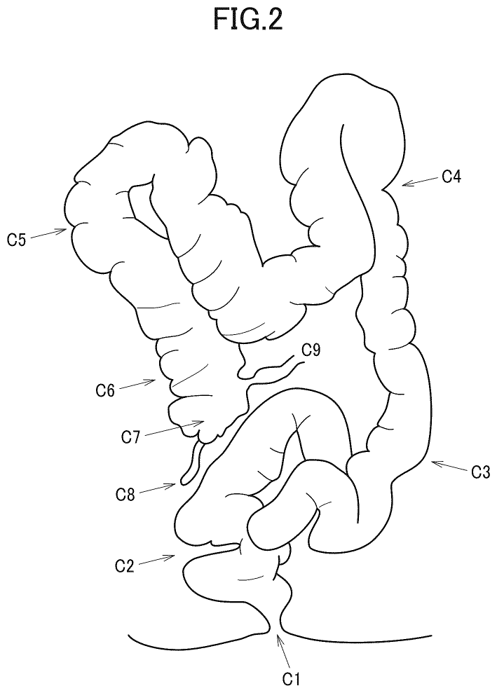

FIG. 2 is a diagram illustrating a large intestine. The large intestine is an intra-abdominal organ continuous from the anus denoted by C1 toward the oral cavity side to the small intestine denoted by C9. Note that the portion from the anus to the position denoted by C2 is called the rectum, the portion from the position denoted by C2 to the position denoted by C3 is called the sigmoid colon, and the portion from the position denoted by C3 to the position denoted by C4 is called the descending colon. Further, as denoted by C3, the boundary between the sigmoid colon and the descending colon is called the SD junction. Further, the portion from the position denoted by C4 to the position denoted by C5 is called the transverse colon, the portion from the position denoted by C5 to the position denoted by C6 is called the ascending colon, the site denoted by C7 is called the cecum, and the site denoted by C8 is called the appendix. In the present embodiment, the side toward which the endoscope 100 advances during large intestine endoscopy is sometimes referred to as inner side. That is, the cecum is on the innermost side of the large intestine, and the anus is on the outermost side thereof. In large intestine endoscopy, a lumen is inflated using gas such as carbon dioxide, and the endoscope 100 is inserted so that the distal end portion 130 to be described later reaches the cecum to perform observation and the like while pulling back the endoscope. Note that, in the subsequent description, inserting the endoscope 100 is sometimes more specifically represented as inserting an insertion portion 110, and these representations are synonymous.

The large intestine endoscopy is an examination with high insertion difficulty o the endoscope 100. Here, high insertion difficulty means that it is difficult to cause the distal end portion 130 of the endoscope 100 to safely reach a desired position. Causing the distal end portion 130 of the endoscope 100 to safely reach means inserting the endoscope 100 in a manner that minimizes pain given to the subject.

One reason for the pain that the subject may feel during large intestine endoscopy is excessive stretching of the mesentery, for example. More specifically, among the portions of the large intestine shown in FIG. 2, the transverse colon and the sigmoid colon are covered by the mesentery and suspended from the abdominal wall and the like via this mesentery. Therefore, the transverse colon and the sigmoid colon have mobility. Accordingly, along with insertion of the endoscope 100, the sigmoid colon and the like are excessively moved by force applied by the insertion portion 110 to be described later to cause excessive stretching of the mesentery, which causes the subject to feel pain via nerves passing through the mesentery. The ascending colon, the descending colon, and the rectum do not have the mesentery and are directly secured to the abdominal wall and the like, and thus do not move. Consequently, the difficulty of inserting the endoscope 100 into the ascending colon, the descending colon, or the rectum is lower than the difficulty of inserting the endoscope 100 into the transverse colon or the sigmoid colon.

The technique using the information processing device 20 according to the present embodiment can be applied to the endoscopic system 10 whose operation is automatically controlled. Here, operation being automatically controlled means that the operation of the endoscope 100 is not a manual operation by a user such as an operator, but an automatic operation by the drive control device 200 or the like to be described later. That is, the endoscope 100 according to the present embodiment is an electric endoscope, and the technique according to the present embodiment is applied to the electric endoscopic system 10. Note that the entity that operates the information processing device 20, the endoscope 100, and the like can be also called an operator. However, in the subsequent description, this entity will be uniformly represented as a user.

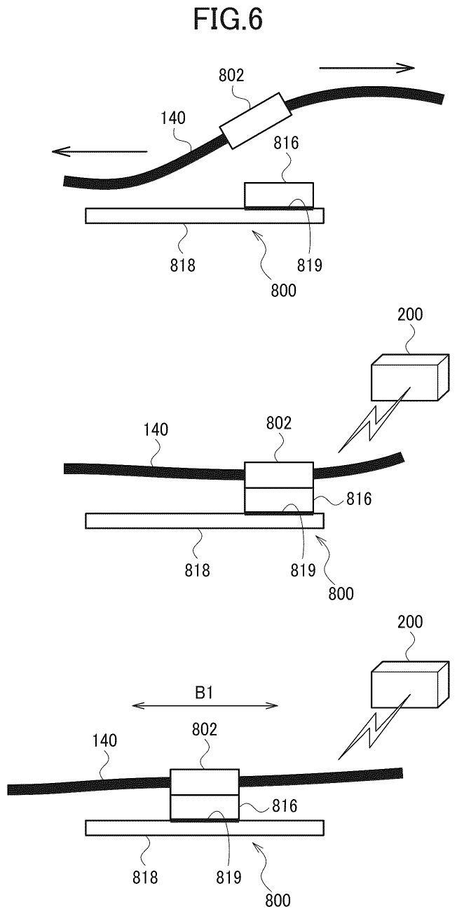

More specifically, automatic control is fully automatic control, for example. but may also be semi-automatic control or partially automatic control. The fully automatic control means that the drive control device 200 performs entire control of the endoscope 100, including a forward/backward movement operation, a bending operation, and a roll rotation operation, which will be described later. Note that forward/backward movement is a simplified expression for forward movement and backward movement. The roll rotation operation in the present embodiment is the operation of rotating the insertion portion 110 with respect to a roll axis, which is the axis of an intracorporeal flexible portion 119 to be described later with reference to FIG. 7. For example, a forward/backward movement direction denoted by B1 to be described later with reference to FIG. 6 is the direction along the roll axis described above. The semi-automatic control means that the drive control device 200 performs one or more, which is not all, of the operations of the endoscope 100. More specifically, the endoscopic system 10 is semi-automatically controlled in a situation where, for example, the bending operation is performed based on the determination by the drive control device 200, while the forward/backward movement operation and the roll rotation operation are performed based on the determination by the user. The partially automatic control means that, for example, the endoscope 100 is operated based on the determination by the user in a situation where the endoscope 100 is inserted without problem, but the drive control device 200 operates the endoscope 100 in a predetermined situation. The predetermined situation means, for example, a situation where a predetermined loop to be described later is formed in the insertion portion 110 to be described later to make it difficult to continue inserting the endoscope 100 depending on the ability of the user.

FIG. 3 shows a detailed example configuration of the electric endoscopic system 10. The electric endoscopic system 10 is not limited to that shown in FIG. 3, and there are many known configurations proposed, to which the technique according to the present embodiment can be widely applied. The endoscopic system 10 shown in FIG. 3 is a system for observing or treating the interior of the subject lying on an operating table T. The endoscopic system 10 includes the endoscope 100, a control device 600, an operation device 300, a treatment tool 400, a forward/backward drive device 800, and a display device 900. The control device 600 includes the drive control device 200 and a video control device 500. The control device 600 in FIG. 3 corresponds to the information processing device 20 in FIG. 1.

The endoscope 100 is a device that is inserted into the lumen of the subject observe the affected site. In the present embodiment, the side inserted into the lumen of the subject is referred to as “distal end side”, and the side attached to the control device 600 is referred to as “proximal end side”. The endoscope 100 includes the insertion portion 110, a joint portion 125, an extracorporeal flexible portion 140, and connectors 201 and 202. The insertion portion 110, the joint portion 125, the extracorporeal flexible portion 140, and the connectors 201 and 202 are coupled to each other in this order from the distal end side.

The insertion portion 110 is a portion inserted into the lumen of the subject an has structure of a flexible, elongated shape. The insertion portion 110 in FIG. 3 includes a bending portion 102, the intracorporeal flexible portion 119 coupling the proximal end of the bending portion 102 to the joint portion 125, and the distal end portion 130 at the distal end of the bending portion 102. An internal path 101 is disposed inside the insertion portion 110, the joint portion 125, and the extracorporeal flexible portion 140. A bending wire 160 to be described later passes through the internal path 101 to be coupled to the bending portion 102. When the drive control device 200 drives the bending wire 160 via the connector 201, the bending portion 102 operates to bend.

An image signal line coupling an imaging device (not shown) included in the distal end portion 130 to the connector 202 passes through the internal path 101. Via this image signal line, an image signal is transmitted from the imaging device to the video control device 500. The video control device 500 displays an endoscopic image generated from the image signal on the display device 900.

The joint portion 125 is provided with an insertion port 190 for the treatment tool 400, and a roll operation portion 121. A treatment tool channel is disposed in the internal path 101, the treatment tool channel having one end with an opening at the distal end portion 130 and the other end having an opening at the insertion port 190 for the treatment tool 400. An extension tube 192 extending from the insertion port 190 to the operation device 300 is coupled to the insertion port 190. The treatment tool 400 is inserted through the opening on the operation device 300 side of the extension tube 192 and extends through the insertion port 190 and the treatment tool channel to protrude to the opening in the distal end portion 130. Note that the extension tube 192 may be omitted and the treatment tool 400 may be inserted through the insertion port 190. The roll operation portion 121 is attached to the joint portion 125 so as to be rotatable about the axial direction of the insertion portion 110. When the roll operation portion 121 is operated to rotate, the insertion portion 110 performs roll rotation. As described later, the roll operation portion 121 can be electrically driven.

As described later in detail with reference to FIG. 6, the forward/backward drive device 800 is a drive device that electrically drives the insertion portion 110 to move forward and backward. The extracorporeal flexible portion 140 is detachable from the forward/backward drive device 800. When the forward/backward drive device 800 slides the extracorporeal flexible portion 140 in the axial direction with the extracorporeal flexible portion 140 being attached to the forward/backward drive device 800, the insertion portion 110 moves forward and backward. FIG. 6 to be described later shows an example in which the extracorporeal flexible portion 140 and the forward/backward drive device 800 are detachable. However, the configuration is not limited to this example, and the joint portion 125 and the forward/backward drive device 800 may be configured to be detachable.

The drive control device 200 drives an actuator such as an internal motor on the basis of control by a drive controller 260 to be described later to electrically drive the endoscope 100. Alternatively, in a case where the actuator is outside the drive control device 200, the drive control device 200 sends a control signal to the external actuator to control the electric driving. The drive control device 200 may also drive an internal pump or the like to cause the endoscope 100 to perform gas supply and suction. The gas supply and suction are performed via a gas supply and suction tube passing through the internal path 101. One end of the gas supply and suction tube has an opening at the distal end portion 130 of the endoscope 100, and the other end is coupled to the drive control device 200 via the connector 201.

The operation device 300 is detachably coupled to the drive control device 200 via an operation cable 301. The operation device 300 may communicate with the drive control device 200 through wireless communication rather than wired communication. For example, in a case where the endoscope 100 is driven semi-automatically or partially automatically as described above, when the user operates the operation device 300, the signal of that operation input is transmitted to the drive control device 200 via the operation cable 301. Then, the drive control device 200 electrically drives the endoscope 100 on the basis of the signal from the operation input so that the endoscope moves according to the operation input. The operation device 300 has multi-channel operating input units corresponding to the forward movement and backward movement, bidirectional bending operation, and roll rotation of the endoscope 100. Each operation input unit is constituted by, for example, a dial, joystick, cross key, button, switch, touch panel, or the like. Note that the operation of moving the insertion portion 110 forward is also referred to as operation of pushing the insertion portion 110, and the operation of moving the insertion portion 110 backward is also referred to as operation of pulling the insertion portion 110. For example, the control to push the endoscope 100 by a predetermined distance and the control to pull the endoscope 100 by a predetermined distance can be collectively referred to as forward/backward movement control. This forward/backward movement control is sometimes called jiggling.

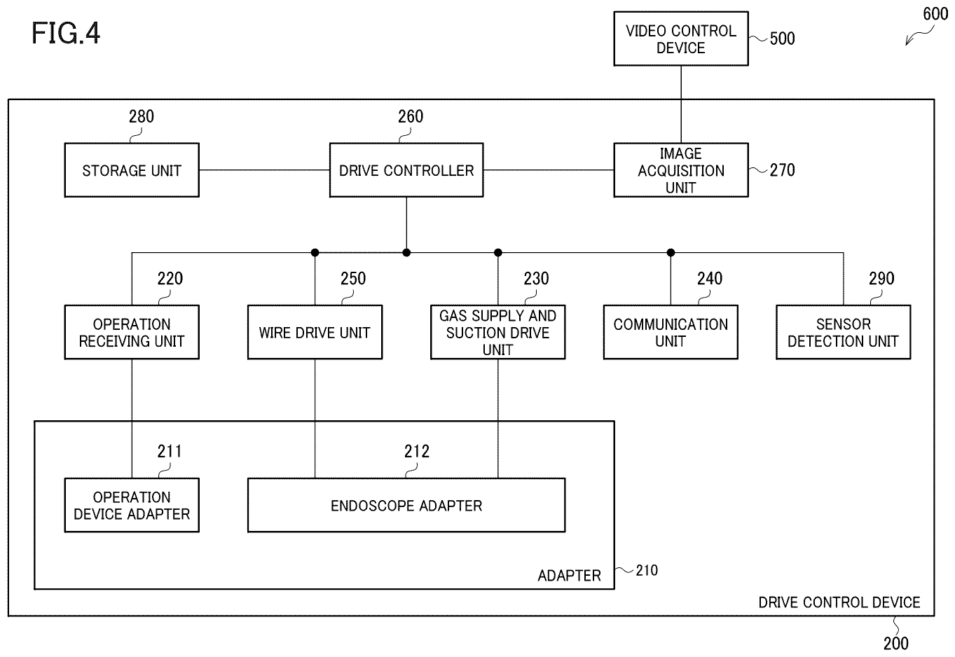

FIG. 4 shows a detailed example configuration of the drive control device 200. The drive control device 200 includes an adapter 210, an operation receiving unit 220, a gas supply and suction drive unit 230, a communication unit 240, a wire drive unit 250, a drive controller 260, an image acquisition unit 270, a storage unit 280, and a sensor detection unit 290.

The adapter 210 has an operation device adapter 211 to which the operation cable 301 is detachably coupled, and an endoscope adapter 212 to which the connector 201 of the endoscope 100 is detachably coupled.

Based on a control signal from the drive controller 260, the wire drive unit 250 drives the bending portion 102 of the endoscope 100 to cause the bending operation thereof. The wire drive unit 250 includes a bending operation motor unit that drives the bending portion 102 of the endoscope 100. The endoscope adapter 212 has a bending operation coupling mechanism for coupling to the bending wire 160 on the endoscope 100 side. When the coupling mechanism is driven by the bending operation motor unit, the driving force thereof is transmitted to the bending wire 160 on the endoscope 100 side.

Based on a control signal from the drive controller 260, the gas supply and suction drive unit 230 drives the endoscope 100 to cause the gas supply and suction thereof. The gas supply and suction drive unit 230 is coupled to the gas supply and suction tube of the endoscope 100 via the endoscope adapter 212. The gas supply and suction drive unit 230 includes a pneumoperitoneum device and the like, and supplies air to the gas supply and suction tube and sucks air from the gas supply and suction tube.

The communication unit 240 performs communication with drive devices disposed outside the drive control device 200. The communication may be either wireless or wired. The drive devices disposed outside are the forward/backward drive device 800 for allowing forward/backward movement, the roll drive device 850 for allowing roll rotation, and the like.

The image acquisition unit 270 is a communication interface that receives image data of endoscopic images from the video control device 500 via wired or wireless communication. For example, based on the endoscopic images acquired from the image acquisition unit 270, the drive controller 260 generates insertion control information to be described later.

The drive controller 260 controls forward/backward movement, a bending operation, and a roll rotation of the endoscope 100, and gas supply and suction by the endoscope 100. The drive controller 260 is hardware corresponding to the processor 22 in FIG. 1. The drive controller 260 controls the electric drive on the basis of a signal of an operation input from the operation receiving unit 220. Specifically, when the bending operation of the bending portion 102 is performed, the drive controller 260 outputs a control signal indicating a bending direction or a bending angle to the wire drive unit 250. Then, the wire drive unit 250 drives the bending wire 160 so that the bending portion 102 bends in that bending direction or at that bending angle. When the forward/backward movement operation is performed, the drive controller 260 sends a control signal indicating a forward/backward movement direction or a forward/backward movement amount to the forward/backward drive device 800 via the communication unit 240. Then, the forward/backward drive device 800 moves the extracorporeal flexible portion 140 forward and backward so that the endoscope 100 moves forward and backward in that forward/backward movement direction or by that forward/backward movement amount. When the roll rotation operation is performed, the drive controller 260 sends a control signal indicating a roll rotation direction or a roll rotation angle to the roll drive device 850 to be described later via the communication unit 240. Then, the roll drive device 850 causes the insertion portion 110 to perform roll rotation in that roll rotation direction or at that roll rotation angle. Similar control is performed for other electric driving.

The sensor detection unit 290, in the case of including a predetermined sensor the like included in the endoscope 100, includes an amplifier circuit that amplifies the output signal from the predetermined sensor, and an A/D converter that performs A/D conversion of the output signal from the amplifier circuit to output detection data to the drive controller 260. If the endoscope 100 does not include the predetermined sensor, the sensor detection unit 290 may be omitted.

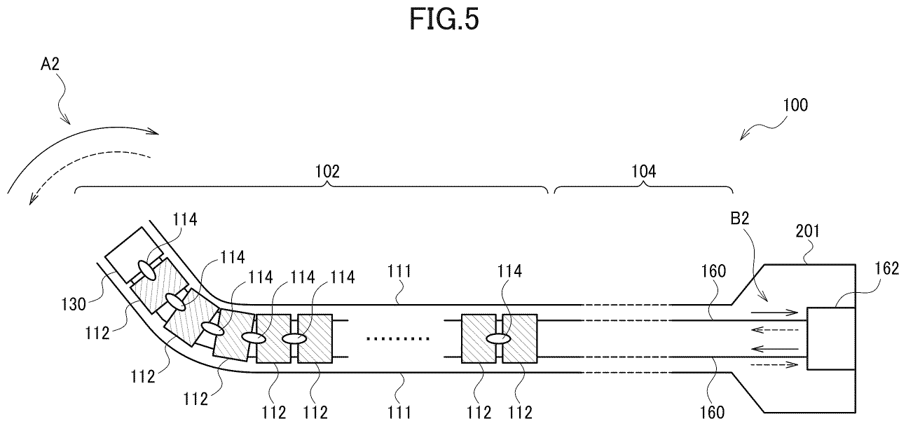

FIG. 5 schematically shows the endoscope 100 including the bending portion 102 and a mechanism to drive the bending portion 102. The endoscope 100 includes the bending portion 102, a flexible portion 104, and the connector 201. Note that the flexible portion 104 corresponds to the intracorporeal flexible portion 119 and the extracorporeal flexible portion 140 described with reference to FIG. 3, and the joint portion 125 is not shown in FIG. 5.

The bending portion 102 and the flexible portion 104 are covered by an outer sheath 111. The tube interior of this outer sheath 111 corresponds to the internal path 101 in FIG. 3. The bending portion 102 includes a plurality of bending pieces 112 and the distal end portion 130 linked to the distal end of the leading bending piece 112. The plurality of bending pieces 112 and the distal end portion 130 are linked to each other by rotatable bending piece joint portions 114 in series from the proximal end side to the distal end side, to thereby form multi-joint structure. The connector 201 is provided with an endoscope-side coupling mechanism 162 to be coupled to the coupling mechanism on the drive control device 200 side. When this connector 201 is attached to the drive control device 200, electrical driving is made possible for the bending operation. Inside the outer sheath 111, the bending wire 160 is disposed. One end of the bending wire 160 is coupled to the distal end portion 130. The bending wire 160 extends through the plurality of bending pieces 112 to pass through the flexible portion 104, turns back in the coupling mechanism 162 to pass through the flexible portion 104 again, and extends through the plurality of bending pieces 112. The other end of the bending wire 160 is coupled to the distal end portion 130. The driving force from the wire drive unit 250 is transmitted via the coupling mechanism 162 to the bending wire 160 as a pulling force of the bending wire 160.

As a solid arrow denoted by B2 shows, when the upper wire in the drawing pulled, the lower wire is pushed, which causes the multi-joints of the bending pieces 112 to bend upward in the drawing. As a result, as a solid arrow denoted by A2 shows, the bending portion 102 bends upward in the drawing. As a dashed arrow denoted by B2 shows, when the lower wire in the drawing is pulled, the bending portion 102 similarly bends downward in the drawing as a dashed arrow denoted by A2 shows. Note that the bending portion 102 can bend independently in two directions orthogonal to each other. FIG. 5 shows a bending mechanism for one direction. However, in practice, two bending wires 160 are provided, and each of the bending wires 160 is independently pulled by the coupling mechanism 162 to enable independent bending in two directions.

Note that the mechanism for electrically performing bending is not limited to the above. For example, a motor unit may be provided in place of the coupling mechanism 162. Specifically, the drive control device 200 may send a control signal to the motor unit via the connector 201, and the motor unit may drive and pull or relax the bending wire 160 on the basis of the control signal to cause the bending operation thereof.

FIG. 6 shows a detailed example configuration of the forward/backward drive device 800. The forward/backward drive device 800 includes a motor unit 816, a base 818, and a slider 819. As shown in the upper diagram and the middle diagram, an attachment 802 detachable from the motor unit 816 is disposed on the extracorporeal flexible portion 140 of the endoscope 100. As shown in the middle diagram, when the attachment 802 is attached to the motor unit 816, electric driving for forward/backward movement is made possible. As shown in the bottom diagram, the slider 819 supports the motor unit 816 so that the motor unit 816 can move linearly to the base 818. This slider 819 is secured to the operating table T shown in FIG. 3. As denoted by B1, the drive control device 200 sends a control signal for forward movement or backward movement to the motor unit 816 via wireless communication. Then, based on the control signal, the motor unit 816 and the attachment 802 move linearly on the slider 819. As a result, the forward movement and backward movement of the endoscope 100 are achieved. Note that the drive control device 200 and the motor unit 816 may be wire-connected.

FIG. 7 is a perspective view showing the joint portion 125 including the roll drive device 850. The joint portion 125 includes a joint portion body 124 and the roll drive device 850. The insertion port 190 for the treatment tool is disposed on the joint portion body 124, and connects with the treatment tool channel inside the joint portion body 124. The joint portion body 124 has a shape of a cylinder, and a cylindrical member coaxial with the cylinder is disposed rotatably inside the joint portion body 124. The proximal end portion of the intracorporeal flexible portion 119 is secured to the outside of the cylindrical member, and the proximal end portion serves as the roll operation portion 121. As a result, the intracorporeal flexible portion 119 and the cylindrical member can rotate about the axial direction of the intracorporeal flexible portion 119 relative to the joint portion body 124. The roll drive device 850 is a motor unit disposed inside the joint portion body 124. As denoted by B3, the drive control device 200 sends a control signal for roll rotation to the roll drive device 850 by wireless communication. Then, based on the control signal, the roll drive device 850 rotates the proximal end portion of the intracorporeal flexible portion 119 relative to the joint portion body 124, so that the intracorporeal flexible portion 119 performs roll rotation. As a result, roll rotation of the endoscope 100 is achieved. Note that the roll drive device 850 may include a clutch mechanism, and the clutch mechanism may switch non-electric roll rotation and electric roll rotation. The drive control device 200 and the roll drive device 850 may also be wire-connected by a signal line passing through the internal path 101.

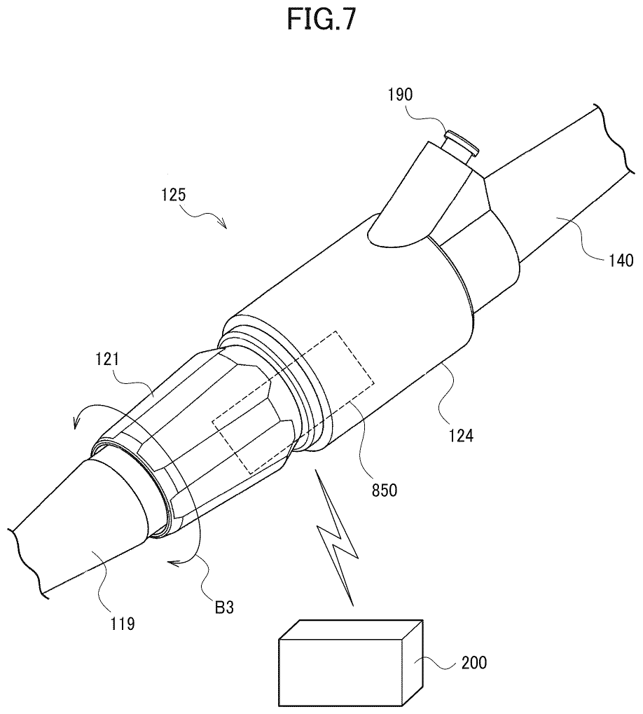

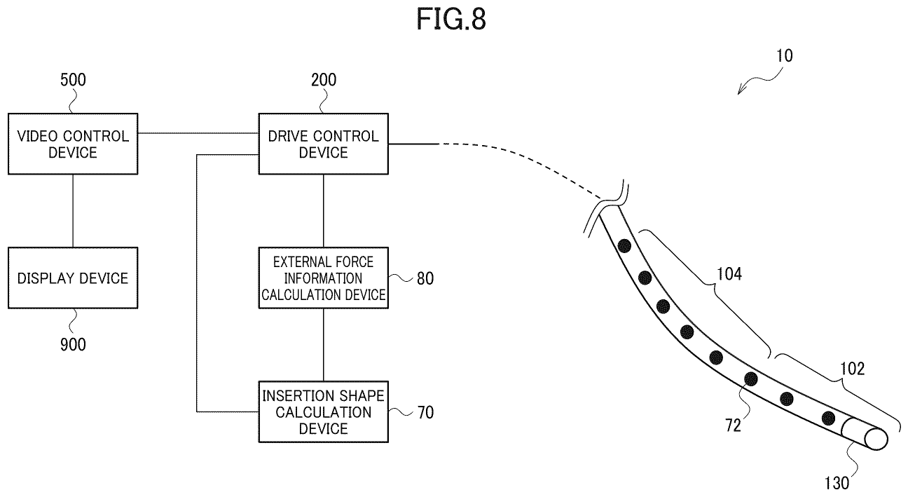

The endoscopic system 10 according to the present embodiment may also perform automatic drive control of the endoscope 100 and may be able to estimate the shape of the insertion portion 110 and the external force acting on the insertion portion 110. Many known methods have been proposed for estimation of the shape of the insertion portion 110. For example, the endoscopic system 10 of FIG. 3 further including an insertion shape calculation device 70, source coils 72, an external force information calculation device 80, and the like shown in FIG. 8 can achieve the estimation of the shape of the insertion portion 110 and the external force acting on the insertion portion 110.

For example, a plurality of source coils 72 are disposed in the insertion portion 110 at predetermined intervals. For example, a current generation device (not shown) sequentially causes the source coils 72 to output a sinusoidal current starting from, for example, the source coil 72 at the distal end side of the insertion portion 110. Each of the source coils 72 generates a magnetic field by the current. The current generation device may be included in the drive control device 200. The insertion shape calculation device 70 detects the magnetic fields generated from the respective source coils 72 via an antenna (not shown) and acquires position information of the respective source coils 72 on the basis of the intensity of the detected magnetic fields. Further, the insertion shape calculation device 70 generates insertion shape information of the insertion portion 110 on the basis of the acquired position information of each of the plurality of source coils 72, and sends the generated insertion shape information to the drive control device 200 and the external force information calculation device 80.

The external force information calculation device 80 calculates information on external forces applied to respective positions of the insertion portion 110 in the longitudinal direction on the basis of the shape information of the insertion portion 110 received from the insertion shape calculation device 70. The memory (not shown) of the external force information calculation device 80 stores in advance, for example, curvature data and curvature angle data of a plurality of predetermined positions of the insertion portion 110 in a state where no external force is applied, and curvature data and curvature angle data of the plurality of predetermined positions of the insertion portion 110 acquired in a state where a predetermined external force is applied to any position of the insertion portion 110 from any conceivable directions. Note that the memory may store, in place of the curvature data, radius of curvature data. The external force information calculation device 80 calculates, based on, for example, various types of received data of the insertion portion 110 at the positions of the respective source coils 72 and various types of stored data, external force information of the positions of the respective source coils 72, and sends the calculated external force information to the drive control device 200. Examples of the external force information include information on the magnitude of the external force and information on the direction of the external force.

Note that the calculation method of the insertion shape is not limited to the method using magnetic fields, and may be a method using, for example, ultrasonic, light, or the like. In this case, for example, although not shown, an ultrasonic sensor, an optical fiber sensor, a strain sensor, or the like may be disposed in the insertion portion 110, and a detection signal from such a sensor may be sent to the sensor detection unit 290 described above, and the sensor detection unit 290 may send detection data to the drive controller 260.

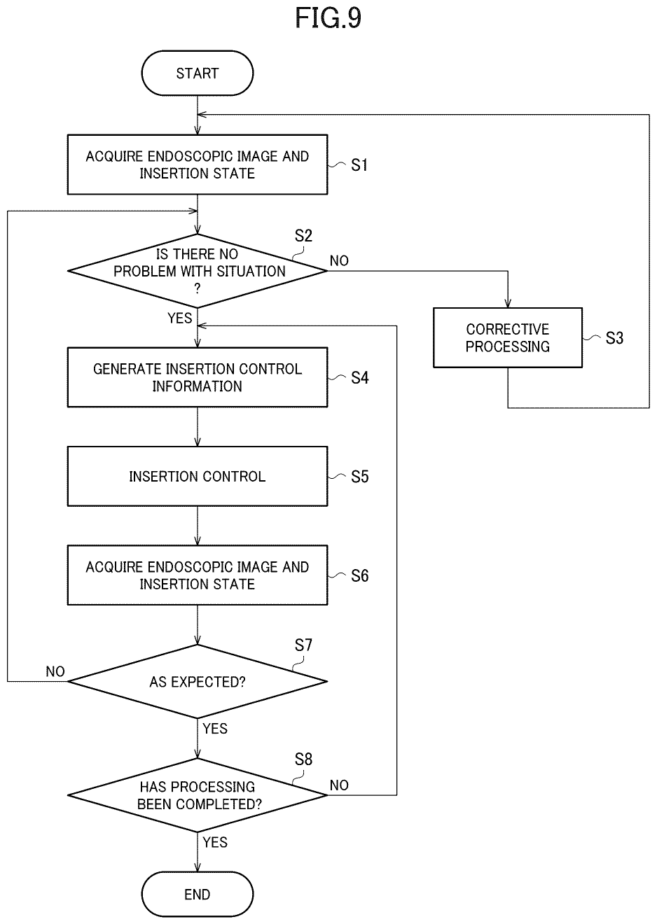

The processing for automatic control of the endoscope 100 can be realized, for example, by the processor 22 performing the technique disclosed in WO 2019/155617. The flowchart of FIG. 9 shows a flowchart of the automatic control in WO 2019/155617 in a simplified manner. For convenience of illustration, the wording in each flow of FIG. 9 is described in a partially simplified manner. The processor 22 acquires an endoscopic image and insertion state information (step S1). Specifically, for example, the drive controller 260 acquires an endoscopic image via the image acquisition unit 270, acquires the insertion shape information from the insertion shape calculation device 70 described above, and acquires the external force information from the external force information calculation device 80 described above. That is, the insertion state in step S1 of FIG. 9 is an abbreviation of the insertion state information, which is specifically the insertion shape information and external force information described above.

Then, the processor 22 determines an insertion situation on the basis of the acquired endoscopic image and insertion state. In the case of determination that there is no problem with the insertion situation (YES in step S2), the processor 22 generates the insertion control information (step S4). On the other hand, in the case of determination that there is a problem with the insertion situation (NO in step S2), the processor 22 performs corrective processing (step S3) and repeats step S1. The corrective processing (step S3) is repeated until determination of YES is made in step S2. That is, the situation in step S2 of FIG. 9 is an abbreviation of the insertion situation. A problem with the insertion situation is, for example, deflection being generated in the insertion portion 110, a predetermined loop to be described later being formed in the insertion portion 110, a force of an amount equal to or greater than a predetermined standard value being applied to the insertion portion 110, a lumen direction being lost, or the like. Note that the technique for making determination to such an insertion situation is known and description of details thereof is omitted. However, for example, whether the predetermined loop is formed is determined based on, for example, whether the shape based on the insertion shape information described above has higher similarity to the shape related to the predetermined loop stored in advance than a predetermined standard value. When, for example, the subject complains of pain, the processor 22 may perform processing to make determination of NO in step S2 as interrupt processing.

The corrective processing (step S3) is a procedure to be taken in a case where determination of NO is made in step S2. That is, various types of programs to deal with the problems described above are stored in the storage unit 280 of the drive control device 200, and the processor 22 identifies the cause of the determination of NO made in step S2, selects and executes the program corresponding to that cause. Since many known techniques for eliminating these causes have been proposed, detailed description thereof is omitted, and examples of the technique are as described below.

For example, in the case of determination made in step S2 that the insertion portion 110 is deflected, the processor 22 performs processing of repeating forward/backward movement control by a predetermined distance a predetermined number of times. Alternatively, the processor 22 may perform processing that is a combination of forward/backward movement control of a predetermined number of times and roll rotation control at a predetermined angle. In this manner, the deflection of the insert portion 110 can be eliminated.

In the case of determination made in step S2 that a predetermined loop is formed in the insertion portion 110, for example, the processor 22 performs processing that is a combination of the forward/backward movement control and the roll rotation control in order to resolve the predetermined loop. Examples of the predetermined loop are an α-loop, an inverse α-loop, an N-loop, a γ-loop, and the like. Although a detailed description is omitted, control programs each including a combination of the forward/backward movement control and the roll rotation control suitable for the respective loops are known for resolving these loops, and these control programs are stored in the storage unit 280. Then, the processor 22 identifies the type of the loop in step S3, selects a suitable control program for resolving the identified loop, and resolves the identified loop. Note that the processor 22 may identify the position of the distal end portion 130 and may make determination of not resolving the loop in a case where the identified distal end portion 130 has reached the inner side of the large intestine. In this manner, appropriate action can be taken against the formation of loops.

In the case of determination made in step S2 that a force of an amount equal to or greater than a predetermined standard value is applied to the insertion portion 110, for example, the same processing is performed as in the case of determination that the insertion portion 110 is deflected. That is, the processor 22 performs processing of repeating the forward/backward movement control by a predetermined distance a predetermined number of times or processing that is a combination of the forward/backward movement control of a predetermined number of times and the roll rotation control at a predetermined angle. In this manner, it is possible to eliminate the excessive resistance received by the insertion portion 110.

In the case of determination made in step S2 that the lumen direction is lost, example, the processor 22 performs processing of, for example, improving the field of view of the imaging device at the distal end portion 130. Examples of the processing of improving the field of view of the imaging device are, for example, processing of searching the lumen thorough curvature control and rotation control, processing of cleaning the surface of an objective lens of the imaging device through water supply control, processing of changing the field of view of the imaging device through control to move the distal end portion 130 backward, processing of expanding the lumen through gas supply control, and the like. In a case where the lumen direction is lost for the reason that the lumen is collapsed due to the concentration of folds, the processor 22 may perform the bending control so as to direct the distal end portion 130 in the direction in which the lumen is collapsed, and then perform the processing of expanding the lumen through the gas supply control. In this manner, the field of view of the imaging device is improved and the lumen direction can be found.

Then, the processor 22 generates the insertion control information (step S4). For example, the processor 22 detects the lumen direction from the acquired endoscopic image and generates a control program to cause the insertion portion 110 to move forward, bend, and perform roll rotation. The technique of detecting the lumen direction from the endoscopic image is not described in detail, and examples of the technique include a technique of evaluating brightness of the acquired endoscopic image and a technique that is a combination of a technique of estimating the relative positional relationship in the depth direction of each pixel and region of the endoscopic image and a technique of dividing the region according to the structure of the intestinal wall, folds, and the like in the endoscopic image.

Although detailed description and illustration are omitted, machine learning may be used to generate the insertion control information. For example, a trained model is stored in the storage unit 280, the trained model having been machine-trained by a data set that uses endoscopic images as input data and operation details as a correct label for the endoscopic images. Then, in step S4, the drive controller 260 infers suitable operation details from the endoscopic image acquired from the image acquisition unit 270 to thereby generate the insertion control information. The trained model includes input layers, output layers, multi-layer neural networks, and the like, and is generated by machine learning such as deep learning. There may be a plurality of types of trained models depending on operational manipulation to be described later. Specifically, the trained model may be separated into a trained model read from the storage unit 280 when a push method to be described later is used as the operational manipulation and a trained model read from the storage unit 280 when a shaft retention shortening method to be described later is used as the operational manipulation. In addition to the application for generating the insertion control information, the trained model may also be used in the application for detecting the lumen direction from the endoscopic image. Specifically, a region dividing method called semantic segmentation can be implemented by using a trained model that includes, for example, a fully convolutional neural network (FCN), and structural information that provides a clue to the lumen can be obtained from the endoscopic image.

Subsequently, the processor 22 performs insertion control on the basis of the insertion control information generated in step S4 (step S5). After step S5 is performed, similarly to step S1, the processor 22 acquires the endoscopic image and the insertion state (step S6).

Then, the processor 22 determines whether the insertion control performed in step S5 has brought the insertion portion 110 into the insertion state as expected. In the case of determination that the insertion unit 110 is in the insertion state as expected (YES in step S7), the processor 22 performs step S8 to be described later. On the other hand, in the case of determination that the insertion portion 110 is not in the insertion sate as expected (NO in step S7), the processor 22 performs step S2. In this case, the processor 22 will make determination of NO in step S2 and perform the corrective processing as described above (step S3).

In a case where determination of YES is made in step S7, the processor 22 determines whether the operation of the endoscope 100 has been completed. In the case of determination that the operation of the endoscope 100 has not been completed (NO in step S8), the processor 22 repeats step S4. As described above with reference to FIG. 2, in large intestine endoscopy, the objective of inserting the endoscope 100 is to make the distal end portion 130 to reach the cecum. Accordingly, the processor 22 determines whether the distal end portion 130 has reached the cecum, which is the innermost side. In a case where the distal end portion 130 has not reached the cecum, the processor 22 makes determination of NO in step S8, and repeats the insertion control of the endoscope 100. On the other hand, in the case of determination that the distal end portion 130 has reached the cecum, the processor 22 determines that operation of the endoscope 100 has been completed (YES in step S8) and ends the flow. In this manner, performing the processing in FIG. 9 can achieve the automatic insertion of the endoscope 100. The control information for insertion of the endoscope 100 in the technique according to the present embodiment is the insertion control information generated in step S4 in the processing of automatic insertion of the endoscope 100 shown in FIG. 9, but the control information may be other information. That is, as described later, in the present embodiment, a condition for generating the insertion control information in step S4 of FIG. 9 are set based on the attribute of the subject. Hereinafter, the control information for insertion of the endoscope 100 may be described simply as control information.

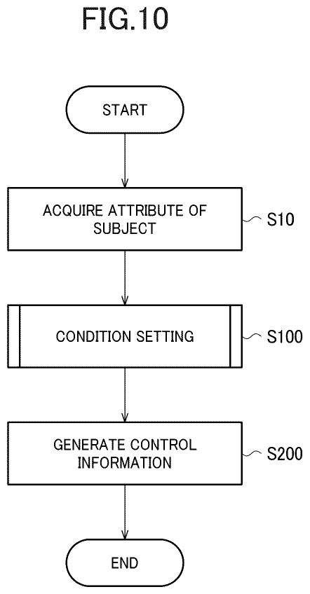

The flowchart of FIG. 10 is used to describe an example of the processing method according to the present embodiment. The processing in FIG. 10 is, for example, processing for the user to activate the information processing device 20 and set the program related to the processing in FIG. 9. The processor 22 acquires the attribute of the subject (step S10). Specifically, for example, step S10 is performed by the user operating the operation unit (not shown) of the information processing device 20 and reading, from a memory (not shown), the attribute of the subject of the endoscopy. Alternatively, step S10 may be performed by a manual input by the user on an operation unit (not shown).

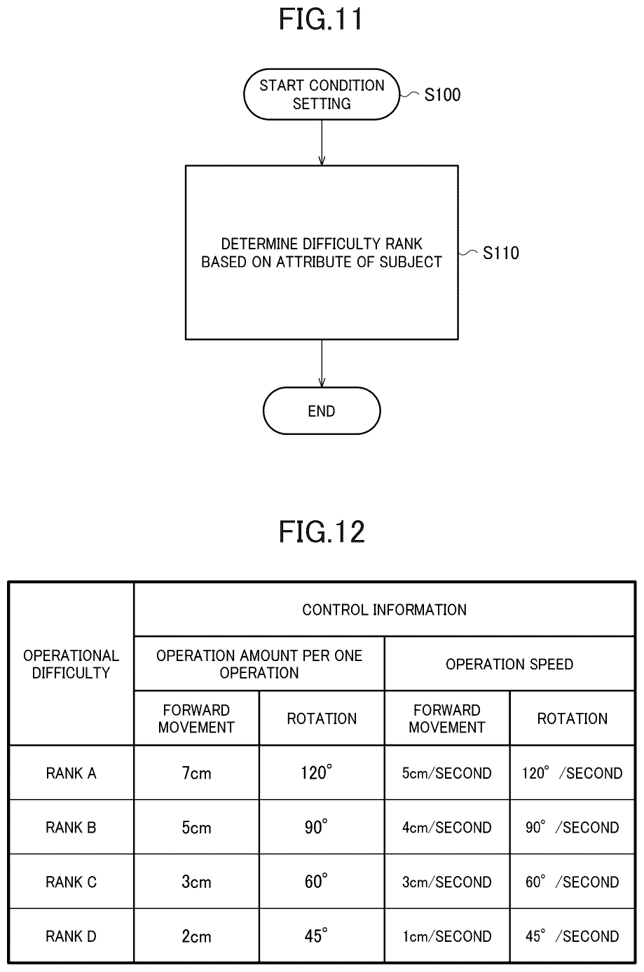

Subsequently, the processor 22 performs condition setting (step S100). More specifically, for example, as shown in the flowchart of FIG. 11, a difficulty rank is determined based on the attribute of the subject (step S110). Examples of the difficulty rank are rank A, rank B, rank C, rank D, and the like, details of which are described later. In the present embodiment, the difficulty rank is classified into four types of rank A, rank B, rank C, and rank D and shown as examples. However, as long as there are a plurality of difficulty ranks to be classified, the difficulty ranks are not limited to four types. The present embodiment shows that the difficulty of inserting the endoscope 100 increases from rank A toward rank D.

Subsequently, the processor 22 generates the control information (step S200). For example, the processor 22 generates the control information on the basis of the condition set by referring to a look-up table of FIG. 12. That is, the processor 22 uses the operational difficulty rank that is the condition set in step S100 as input data to generate the control information as output data. In the following description, the look-up table is simply described as a table. FIG. 12 shows an example in which the rank is set by referring to the table to output the control information. However, without being limited to this example, the ranks to the attributes may be set by sequences such as conditional branching and the like. The sequences such as conditional branching can be implemented by, for example, programming using basic syntax based on programming languages. Examples of the basic syntax are IF statement, THEN statement, ELSE statement, SWITCH statement, and the like. The rank may also be set using, for example, a discriminator created by machine learning. Specifically, for example, the rank may be set using a discriminator created by learning using gender, age, height, and the like as feature amounts and the rank to be set as a class. The feature amounts here are also called feature vectors. In this case, in gender, for example, the feature amounts having variables of 0 as male and 1 as female may be used. A discriminator for males and a discriminator for females may also be provided separately, so that the discriminator can be selected as appropriate depending on the gender of the subject. A plurality of discriminators may also be prepared depending on attributes other than gender. In the table of FIG. 12, the control information is shown to include both the operation amount per one operation and the operation speed, but it suffices that the control information includes at least one of the operation amount per one operation and the operation speed.

In the table of FIG. 12, for example, in a case where the rank of the operational difficulty is rank A, the operation amount per one operation to move forward is 7 cm, the operation amount per one operation to rotate is 120°, the operation speed to move forward is 5 cm/second, and the operation speed to rotate is 120°/second. In a case where the operational difficulty is rank B, the operation amount per one operation to move forward is 5 cm, the operation amount per one operation to rotate is 90°, the operation speed to move forward is 4 cm/second, and the operation speed to rotate is 90°/second. In a case where the operational difficulty is rank C, the operation amount per one operation to move forward is 3 cm, the operation amount per one operation to rotate is 60°, the operation speed to move forward is 3 cm/second, and the operation speed to rotate is 60°/second. In a case where the operational difficulty is rank D, the operation amount per one operation to move forward is 2 cm, the operation amount per one operation to rotate is 45°, the operation speed to move forward is 1 cm/second, and the operation speed to rotate is 45°/second. In this manner, in the table of FIG. 12, both the operation amount and operation speed are shown to vary depending on the operational difficulty ranks, but it suffices that at least one of the operation amount and operation speed varies depending on the operational difficulty rank.

As described above, the processor 22 according to the present embodiment corresponds to the drive controller 260 in the specific endoscopic system 10. That is, it can be said that the processing in FIG. 10 is performed by the drive controller 260. The control information generated in step S200 is more specifically parameters that control the drive control device 200 that electrically drives and controls the endoscope 100.

In this manner, the information processing device 20 according to the present embodiment includes the processor 22 that includes hardware. The processor 22 acquires the attribute of the subject, sets, based on the acquired attribute of the subject, the condition regarding the control information for insertion of the endoscope 100, and generates, based on the set condition, the control information.

In this manner, the information processing device 20 according to the present embodiment can set, before performing endoscopy, a specific condition for generating the control information for insertion of the endoscope 100 on the basis of the acquired attribute of the subject. As a result, more specific control information can be generated. Thus, in endoscopy with high insertion difficulty, such as large intestine endoscopy, it is possible to control the endoscope 100 in more consideration of the subject. In a case where it is the first time for the subject to undergo endoscopy, information known in advance is attribute information of the subject, whereas a specific technique of setting parameters for operating the endoscope from such information has not been proposed. The technique disclosed in WO2016/135966 can be applied only to a subject having an experience of endoscopy in the past.

The technique according to the present embodiment may also be implemented as the endoscopic system 10. That is, the endoscopic system 10 according to the present embodiment includes the information processing device 20 described above and the endoscope 100. With this configuration, the same effect as above can be attained.

The technique according to the present embodiment may also be implemented as a processing method. The processing method according to the present embodiment performs processing of acquiring the attribute of the subject (step S10), processing of setting, based on the acquired attribute of the subject, a condition regarding the control information for insertion of the endoscope 100 (step S100), and processing of generating, based on the set condition, the control information (step S200). In this manner, the same effect as above can be attained.

In the information processing device 20 according to the present embodiment, the processor 22 may generate control information regarding at least one of the operation amount and operation speed of the endoscope 100. In this manner, it is possible to generate more specific control parameters regarding insertion of the endoscope 100.

In the information processing device 20 according to the present embodiment, the processor 22 may generate control information in which at least one of the operation amount and operation speed varies depending on the set condition. In this manner, it is possible to generate specific control parameters regarding insertion of the endoscope 100 in accordance with the attribute of the subject.

In the information processing device 20 according to the present embodiment, the processor 22 may generate, based on the set condition, control information for controlling the drive device that drives insertion of the endoscope 100. In this manner, it is possible to drive and control the endoscope 100 in accordance with the attribute of the subject.

In the information processing device 20 according to the present embodiment, the processor 22 may set, based on the attribute of the subject, the rank of the operational difficulty as a condition, and generate, based on the set condition, the control information. In this manner, it is possible to generate a plurality of types of control information in accordance with the degrees of operational difficulty.

In the information processing unit 20 according to the present embodiment, in the case of determination that the operational difficulty is high, the processor 22 may perform control such that at least one of the operation amount and operation speed as control information is reduced compared with the case of determination that the operational difficulty is low. In this manner, it is possible to perform insertion of the endoscope 100 more carefully for a subject with high operational difficulty.

For example, although no table is shown, an allowable value of a resistance force generated between the insertion portion 110 and the intestinal tract when the insertion unit 110 is moved forward may be used as the control information. For example, in a case where the condition set in step S100 is rank A, the allowable resistance force is set as 7N. Similarly, in a case where the condition set in step S100 is rank B, the allowable resistance force is set as 6N. Similarly, in a case where the condition set in step S100 is rank C, the allowable resistance force is set as 4N. Similarly, in a case where the condition set in step S100 is rank D, the allowable resistance force is set as 3N. For example, in a case where a resistance force of 5N is detected when the endoscope 100 is inserted for a subject set as rank D as a condition and the insertion portion 110 is moved forward, the processor 22 makes determination of NO in step S2 of FIG. 9 as interrupt processing. As a result, the processor 22 executes step S3 of FIG. 9 and executes a control program to mitigate the contact between the intestinal tract and the insertion portion 110. As a result, based on the attribute of the subject, it is possible to perform control of the endoscope 100 in consideration of the burden on the subject.

For example, the technique according to the present embodiment may be applied by partially automatic control depending on the position in the large intestine. For example, in a case where the distal end portion 130 is located in the transverse colon or the sigmoid colon, the endoscope 100 may be controlled by the processing in FIG. 10. In a case where the distal end portion 130 is located in the ascending colon, the descending colon, or the rectum, the user may manually operate the endoscope 100. This is because, as described above in FIG. 2, a scene with high insertion difficulty in large intestine endoscopy is a scene of passing the insertion portion 110 through the transverse colon or the sigmoid colon.

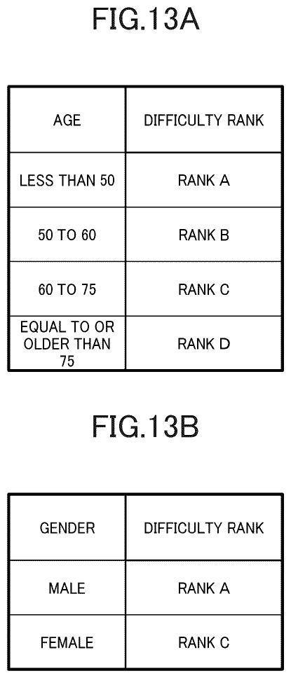

Next, the specific technique for step S110 of FIG. 11 is described in more detail. For example, the processor 22 refers to the table shown in FIG. 13A in a case where the attribute of the subject acquired in step S10 is age. In a case where the age of the subject is less than 50, the processor 22 sets rank A as the difficulty rank by step S110. Similarly, in a case where the age of the subject is 50 or older and less than 60, the processor 22 sets rank B as the difficulty rank by step S110. Note that “50 TO 60” shown in the table of FIG. 13A indicates the age of 50 or older and less than 60. The same applied to other representations. Similarly, in a case where the age of the subject is 60 or older and less than 75, the processor 22 sets rank C as the difficulty rank by step S110. Similarly, in a case where the age of the subject is 75 or older, the processor 22 sets rank D as the difficulty rank by step S110.

That is, FIG. 13A shows that the insertion difficulty of the endoscope 100 is higher in a case where the age of the subject is older than in a case where the age of the subject is younger. The table of FIG. 13A is set taking into consideration that the strength and flexibility of the large intestine tissues of the subject, the condition of the intestinal tract, and the like change with age, and in general, the older age the subject is, the more brittle and less flexible the intestinal tract tends to become. Therefore, it is appropriate to control the endoscope 100 such that at least one of the operation amount and the operation speed is reduced for an older subject compared with a younger subject. Thus, in the information processing device 20 according to the present embodiment, the processor 22 performs control such that at least one of the operation amount and operation speed as control information is reduced in a case where the age, which an attribute of the subject, is older compared with a case where the age is younger. In this manner, it is possible to control the endoscope 100 with or at an appropriate operation amount or operation speed depending on the age of the subject.

For example, the processor 22 may also refer to the table shown in FIG. 13B in a case where gender is acquired as an attribute of the subject in step S10. In a case where the gender of the subject is male, the processor 22 sets rank A as the difficulty rank by step S110. Similarly, in a case where the gender of the subject is female, the processor 22 sets rank C as the difficulty rank by step S110.

That is, FIG. 13B shows that the insertion difficulty of the endoscope 100 is higher in a case where the gender of the subject is female than a case where the gender of the subject is male. The table of FIG. 13B is set taking into consideration that the shape of the pelvis, the fixation situation of the large intestine, an amount of fat in the abdominal cavity, and the like differ between males and females, and that such differences are supposed to be causes of the higher insertion difficulty in large intestine endoscopy for females than for males. More specifically, for example, it tends to be more common in females than in males that the sigmoid colon is depressed in the pelvis, the intestinal tract tends to move excessively due to a low amount of fat in the abdominal cavity, and the like. Therefore, it is appropriate to control the endoscope 100 such that at least one of the operation amount and the operation speed is reduced for a female subject compared with a male subject. Thus, in the information processing device 20 according to the present embodiment, the processor 22 performs control such that at least one of the operation amount and operation speed as control information is reduced in a case where the gender, which an attribute of the subject, is female compared with a case where the gender is male. In this manner, it is possible to control the endoscope 100 with or at an appropriate operation amount or operation speed depending on the gender of the subject.

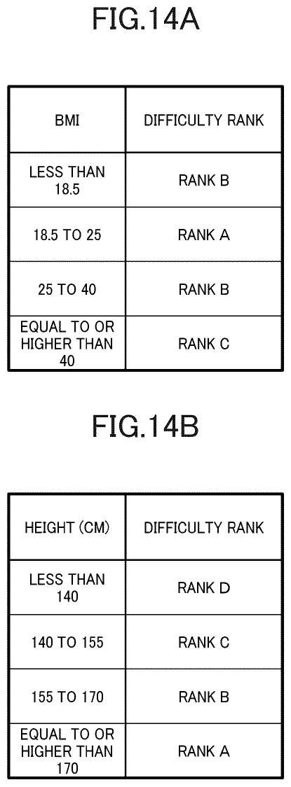

For example, the processor 22 may also refer to the table shown in FIG. 14A in a case where BMI is acquired as an attribute of the subject in step S10. In a case where the BMI of the subject is less than 18.5, the processor 22 sets rank B as the difficulty rank by step S110. In a case where the BMI of the subject is equal to or higher than 18.5 and less than 25, the processor 22 sets rank A as the difficulty rank by step S110. In a case where the BMI of the subject is equal to or higher than 25 and less than 40, the processor 22 sets rank B as the difficulty rank by step S110. In a case where the BMI of the subject is equal to or higher than 40, the processor 22 sets rank C as the difficulty rank by step S110. The table shown in FIG. 14A is set in consideration of the following circumstances. A subject with a high BMI is obese, and therefore have a large amount of visceral fat, which makes it difficult to move the intestinal tract. As a result, for example, in inserting the insertion portion 110 into a strongly bent site, a technique that can reduce pain by operating the insertion portion 110 so as to make the bending portion dull cannot be applied to the subject, which results in high possibility that the subject feels pain during large intestine endoscopy. In addition, a subject with too low BMI tends to have difficulty in inserting the insertion portion 110 due to the complex running form of the intestinal tract.

For example, the processor 22 may also refer to the table shown in FIG. 14B in a case where height is acquired as an attribute of the subject in step S10. In a case where the height of the subject is less than 140 cm, the processor 22 sets rank D as the difficulty rank by step S110. In a case where the height of the subject is equal to or higher than 140 cm and less than 155 cm, the processor 22 sets rank C as the difficulty rank by step S110. In a case where the height of the subject is equal to or higher than 155 cm and less than 170 cm, the processor 22 sets rank B as the difficulty rank by step S110. In a case where the height of the subject is equal to or higher than 170 cm, the processor 22 sets rank A as the difficulty rank by step S110. The table shown in FIG. 14B is set based on the fact that subjects having a small stature have a complex running form of the intestinal tract compared with subjects having a large stature and therefore tends to have difficulty in inserting the insertion portion 110.

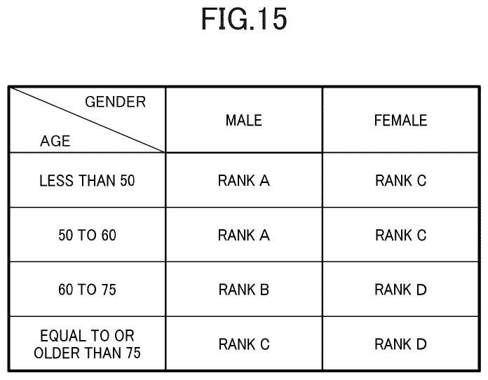

FIGS. 13 and 14 show examples of cases where one attribute of the subject is acquired. However, without being limited to such examples, at least two attributes may be acquired in step S10. In a case where a plurality of attributes are acquired individually, a rank that requires the most careful insertion among them may be set separately. Then, the priority orders may be set for the respective attributes, such as gender and age, and a rank for the attribute with the highest priority order may be adopted. In order to enable appropriate determination in situations where a plurality of attributes are acquired, a multi-dimensional table based on a plurality of attributes may be referenced. For example, FIG. 15 is a two-dimensional table in which a combination of two attributes is associated with the difficulty rank.

In FIG. 15, for example, in a case where the subject is male and his age is less than 50, the processor 22 sets rank A as the difficulty rank by step S110. In a case where the subject is male and his age is equal to or older than 50 and less than 60, the processor 22 sets rank A as the difficulty rank by step S110. In a case where the subject is male and his age is equal to or older than 60 and less than 75, the processor 22 sets rank B as the difficulty rank by step S110. In a case where the subject is male and his age is equal to or older than 75, the processor 22 sets rank C as the difficulty rank by step S110.

In FIG. 15, for example, in a case where the subject is female and her age is less than 50, the processor 22 sets rank C as the difficulty rank by step S110. In a case where the subject is female and her age is equal to or older than 50 and less than 60, the processor 22 sets rank C as the difficulty rank by step S110. In a case where the subject is female and her age is equal to or older than 60 and less than 75, the processor 22 sets rank D as the difficulty rank by step S110. Similarly, in a case where the subject is female and her age is equal to or older than 75, the processor 22 sets rank D as the difficulty rank by step S110.

FIG. 15 shows an example of acquiring age and gender as attributes. However, without being limited to this example, the processor 22 may acquire other combination of attributes, such as acquiring age and height of the subject in step S10. For example, the processor 22 may acquire three or more attributes, such as acquiring age, gender, and height in step S10. In this case, a multi-dimensional table may be stored in advance in the storage unit 280 or the like, the multi-dimensional table including a combination of attributes to be acquired. Consequently, in the information processing device 20 according to the present embodiment, the processor 22 acquires at least two attributes of the subject from age, gender, height, weight, and BMI. In this manner, it is possible to more accurately generate control parameters of the endoscope 100 suitable for the subject.

As attributes of the subject, FIGS. 13 and 14 show age, gender, BMI, and height, which are merely examples, and the attributes of the subject are not limited to them. For example, although not shown in the drawings, in a case where the subject has a history of abdominal surgery, rank C may be set as the difficulty rank by step S110, and in a case where the subject has no history of abdominal surgery, rank A may be set as the difficulty rank by step S110. Subjects having a history of abdominal surgery sometimes have adhesion of the large intestine to surrounding tissues. This may cause the large intestine to have an unordinary form or often cause pain at the site of adhesion. Therefore, it is considered that subjects having a history of abdominal surgery generally have higher insertion difficulty than subjects having no history of abdominal surgery. An interview may also be conducted on the subject about whether he/she can tolerate pain that occurs during endoscopy, and the answer about the pain from the interviewed subject may be used as an attribute of the subject. For example, in a case where the subject answers that he/she can tolerate pain, rank A is set as the difficulty rank by step S110. In a case where the subject answers that he/she has moderate tolerance to pain, rank B is set as the difficulty rank by step S110. In a case where the subject answers that he/she cannot tolerate pain, rank C is set as the difficulty rank by step S110. The family history of the subject may also be included in the attributes of the subject. For example, in a case where one of family members of the subject has had colorectal cancer, it is required more careful endoscopic observation of the large intestine be performed on the subject. Information on the pre-existing condition of the subject may also be used as an attribute of the subject. For example, in a case where the subject has a pre-existing condition of irritable bowel syndrome (IBS) or inflammatory bowel disease (IBD), it is required more careful endoscopic observation be performed on the subject.

In this manner, by further considering the above-described attributes for subjects who are considered to have low operational difficulty in terms of age, gender, or the like, it is possible to clarify that it is appropriate to treat such subjects in the same manner as subjects having high operational difficulty. As a result, it is possible to generate more appropriate control information for the subject.

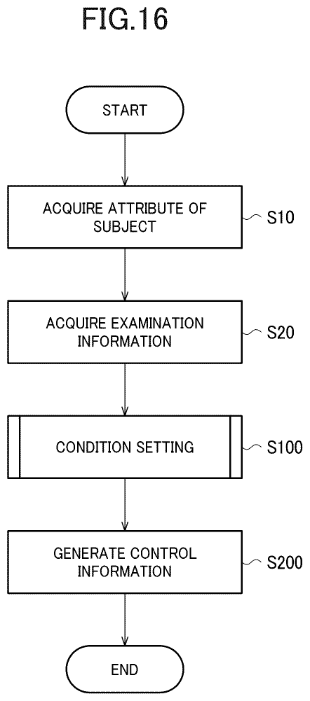

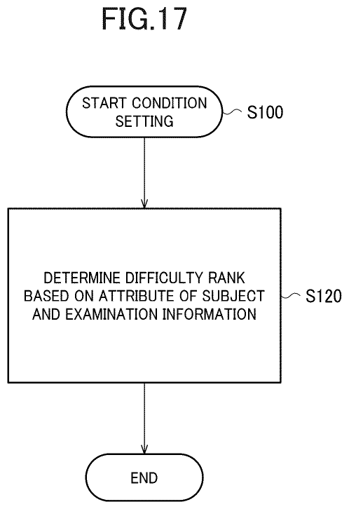

The processing method according to the present embodiment may also be as the example processing shown in the flowchart of FIG. 16, for example. The example processing in FIG. 16 differs from the example processing in FIG. 10 in that, after step S10 described above is performed, examination information is further acquired (step S20). The condition setting (step S100) in FIG. 16 differs from that in FIG. 11 in that it is performed as, more specifically, the flowchart of FIG. 17. In FIG. 17, the processor 22 determines the difficulty rank on the basis of the attribute and examination information of the subject (step S120).

The examination information in step S20 and the like is information regarding examination. The information regarding the examination is the purpose of the examination, for example. The purpose of the examination corresponds to, for example, the reason for having large intestine endoscopy. The examples of the reason are the presence of symptoms such as the hemorrhage described above, the positive result in the fecal occult blood test in the medical examination described above, and the like, and may be treatment such as polyp removal.

For example, the information regarding the examination may also include information regarding sedation. The sedation means inducing a decrease in the level of consciousness by medication. The information regarding sedation includes information regarding the sedation effect and various information to determine whether to combine endoscopy with sedation. In a case where endoscopy and sedation are combined, the subject is made rest for a certain period of time on a bed for recovery or the like after the endoscopy is completed, and thereafter the subject leaves the facility. In a case where endoscopy and sedation are combined, predetermined activities are restricted on the day the endoscopy is performed. Examples of the predetermined activities are driving a car, working at a high place, precision work, and the like.

The sedation effect may be appropriately determined by the user on the basis classification of sedation levels such as minimal sedation, moderate sedation, deep sedation, and general anesthesia. Note that the moderate sedation is also referred to as conscious sedation. The sedation level of moderate sedation is the level at which the subject can respond intentionally to a question from the user, the spontaneous breathing and cardiovascular function of the subject are maintained, and treatment to secure a clear airway for the subject is not required. It is generally considered that the sedation combined with endoscopy requires decreasing pain of the subject, bringing the subject into a state of capable of following instructions from the user, being less accidentalness, and providing a shorter recovery time after sedation is performed. It is considered that the sedation level that meets the above requirements is mainly the moderate sedation. Note that the sedation level of minimal sedation is the level at which the subject can respond normally to a question from the user, and the airway, spontaneous breathing, and cardiovascular function are normal.

For example, the user can set the sedation level equivalent to moderate sedation as “strong sedation effect” and the sedation level equivalent to minimal sedation as “weak sedation effect”. Alternatively, the user may set “strong sedation effect” to a case where the sedation level is moderate sedation and the recovery time after sedation is long, and set “weak sedation effect” to a case where the sedation level is moderate sedation and the recovery time after sedation is short.

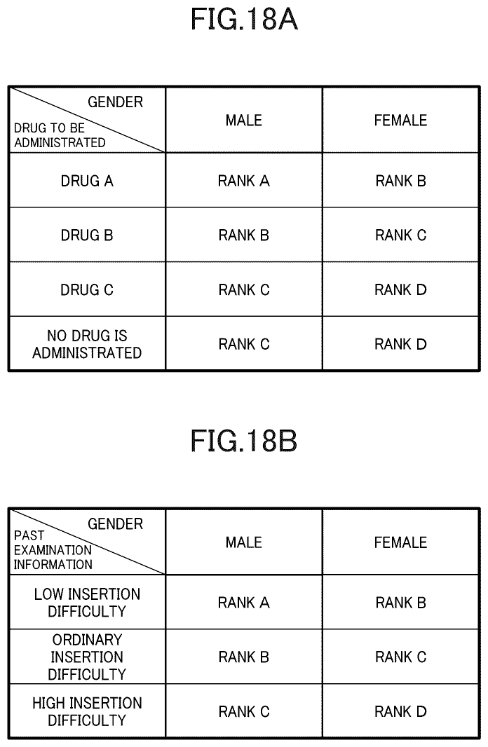

For example, the processor 22 can refer to a table shown in FIG. 18A to attain the same operational effect as the processing of step S110. FIG. 18A shows, as an example, a table based on the combination of gender as a subject attribute and a drug to be administrated. However, without being limited to this combination, the table may be based on the combination of age as a subject attribute and a drug to be administrated. The user may determine the combination as appropriate. In FIG. 18A, in a case where the gender is male, rank A is set as the condition when a drug A is administrated, rank B is set as the condition when a drug B is administrated, rank C is set as the condition when a drug C is administrated, and rank C is set as the condition when no drug is administrated. On the other hand, in a case where the gender is female, rank B is set as the condition when the drug A is administrated, rank C is set as the condition when the drug B is administrated, rank D is set as the condition when the drug C is administrated, and rank D is set as the condition when no drug is administrated. As described above, it is considered that females generally have higher insertion difficulty than males. Therefore, the table of FIG. 18A is set such that the rank set in a case where the same drug is administrated varies depending on the gender.

In FIG. 18A, the drug A is, for example, a drug with a higher sedation effect, and the drug B is, for example, a drug with a lower sedation effect than the drug A. The specific drug may be determined by the user as appropriate in accordance with the set sedation effect. For example, in a case where the sedation level of moderate sedation is set as “strong sedation effect” and the sedation level of minimal sedation is set as “weak sedation effect”, for example, Dolmicum or the like used for moderate sedation may be the drug A and triclofos sodium or the like used for minimal sedation may be the drug B. Alternatively, in a case where the sedation level is moderate sedation and the recovery time after sedation is long is set as “strong sedation effect”, and a case where the sedation level is moderate sedation and the recovery time after sedation is short is set as “weak sedation effect”, Dolmicum may be the drug A and propofol may be the drug B. In FIG. 18A, for both males and females, the condition in the case of administering the drug A having a strong sedation effect is set to have a lower operational difficulty rank than the condition in the case of administering the drug B having a weak sedation effect. That is, in the information processing device 20 according to the present embodiment, in a case where a drug having a strong sedation effect is administered, the processor 22 performs control such that at least one of the operation amount and operation speed as the control information is increased compared with a case where a drug having a weak sedation effect is administered. In this manner, it is possible to control the endoscope 100 with or at an appropriate operation amount or operation speed depending on the strength of the sedation effect of the drug administrated to the subject.

Note that the drug C is, for example, a drug having no sedation effect but having a pain relief effect, which is specifically, for example, pethidine hydrochloride. The pain relief means reducing pain without causing a decrease in the level of consciousness. As described later. in a case where sedation is not combined with endoscopy, the drug C is sometimes administered to the subject under agreement between the user and the subject. In FIG. 18A, since the drug C has no sedation effect, the condition set to the case where the drug C is administered is the same as the condition set to the case where no drug is administered. However, the condition may be different from the condition set to the case where no drug is administered. Although not shown in the drawing, the condition in the case of administering the drug C to the subject in combination with the drug A or the drug B may be the same as the condition for the drug A or the drug B. This is because a sedation effect is produced. In FIG. 18A, for both males and females, the conditions in the cases of administering the drug A and drug B having a sedation effect are set to have a lower rank than the conditions in the cases of not administering the drug A and drug B having a sedation effect. In the information processing device 20 according to the present embodiment, in a case where a drug having a sedation effect is administered, the processor 22 performs control such that at least one of the operation amount and operation speed as the control information is increased compared with a case where such a drug is not administered. In this manner, it is possible to control the endoscope 100 with or at an appropriate operation amount or operation speed depending on the presence of the sedation effect of the drug administrated to the subject.

Note that whether to combine endoscopy and sedation is determined based on the subject's will and consent after the subject has received a sufficient explanation from the user and has satisfied with the explanation. For example, in a case where the subject wants to prioritize eliminating affliction, a sedation level having a strong sedation effect is set. For example, in a case where the subject wishes for sedation to the extent that eliminates anxiety, a sedation level having a weak sedation effect is set. For example, in a case where the subject wishes to observe the endoscopic image with the user, the user sets information that sedation is not to be performed as information regarding sedation. For example, in a case where the subject wishes to minimize the time required for the examination, information that sedation is not to be combined with endoscopy may be set as information regarding sedation. This is because combination of sedation with endoscopy requires time for recovery, which does not meet the wish of the subject.

For example, the information regarding sedation may also include a predetermined geographical circumstance. The predetermined geographical circumstance is a circumstance which requires consideration of whether to combine endoscopy and sedation when, for example, a facility where the endoscopy is performed is located in a place to which the subject himself/herself goes by a private car. More specifically, for example, in a case where the subject himself/herself visits the place by a private car and wishes to drive the private car to go home after the endoscopy, information that sedation is not to be combined with endoscopy may be set as the information regarding sedation. In a case where the technique according to the present embodiment is applied to endoscopy performed in a facility having such a predetermined geographical circumstance, for example, the information that sedation is not to be combined with endoscopy may be set for all subjects as the information regarding sedation. However, in a case where the subject is accompanied by a person who drives a private car instead of the subject, or in a case where the subject uses a taxi or the like for going and returning, individual setting change may be allowed for the information that sedation is to be combined with endoscopy as the information regarding sedation.

In a case where there is no recovery room in the facility where endoscopy is performed, for example, the information that sedation is not to be combined with endoscopy may be set for all subjects as the information regarding sedation. In a case where there is a period when a recovery room temporarily cannot be arranged due to a predetermined circumstance, the information that sedation is not to be combined with endoscopy may be set for all subjects as the information regarding sedation. The predetermined circumstance is for example, a circumstance where dedicated sickrooms must be secured in response to a large number of admission requests for the hospital due to a predetermined epidemic disease. Consequently, in the information processing device 20 according to the present embodiment, the processor 22 acquires examination information including the sedation information that is information regarding sedation, and sets a condition regarding the control information on the basis of the attribute and examination information of the subject. In this manner, it is possible to generate more appropriate control parameters of the endoscope 100 depending on the attribute of the subject and the sedation information.

For example, the information regarding the examination may be information in past large intestine endoscopy. An example of the information in past large intestine endoscopy is information recorded in past large intestine endoscopy, such as information regarding the date and time of the examination, information regarding insertion difficulty, information regarding the type of the endoscope 100 used, information regarding the time required for the examination, and information regarding the manipulation used for the examination.

In this case, for example, in step S120 of FIG. 17, the processor 22 may refer to a table shown in FIG. 18B. In FIG. 18B, in a case where the subject is male and has a record of low insertion difficulty as past examination information, rank A is set as the condition. In a case where the subject is male and has a record of ordinary insertion difficulty as the past examination information, rank B is set as the condition. In a case where the subject is male and has a record of high insertion difficulty as the past examination information, rank C is set as the condition. In a case where the subject is female and has a record of low insertion difficulty as the past examination information, rank B is set as the condition. In a case where the subject is female and has a record of ordinary insertion difficulty as the past examination information, rank C is set as the condition. In a case where the subject is female and has a record of high insertion difficulty as the past examination information, rank D is set as the condition. Note that in FIG. 18B, for the same reasons as in FIG. 18A and the like, in the case of common insertion difficulty as the past examination information, different ranks are set for males and females. Consequently, in the information processing device 20 according to the present embodiment, the processor 22 acquires examination information that is information regarding an examination (step S20), and sets a condition regarding the control information on the basis of the attribute and examination information of the subject (step S120). In this manner, it is possible to generate more appropriate control parameters of the endoscope 100 depending on the attribute of the subject and the examination information.

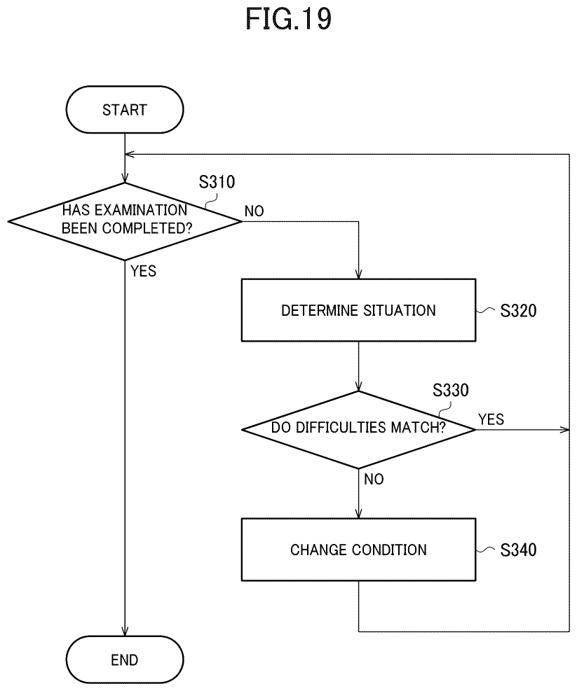

Although the processing shown in FIGS. 10 and 16 is processing for generating control information before large intestine endoscopy, the generated control information may be changed after large intestine endoscopy is started. For example, by periodically performing the example processing shown in the flowchart of FIG. 19 by timer interrupt or the like since large intestine endoscopy has been started, changing the control information at an appropriate timing can be achieved.

In FIG. 19, the processor 22 determines whether large intestine endoscopy has been completed. In the case of determination that large intestine endoscopy has not been completed (NO in step S310), the processor 22 performs processing in step S320 and subsequent steps. Then, the processor 22 determines the insertion situation (step S320). For example, the processor 22 determines whether the number of times of determination of NO made in step S2 or the number of times of determination of NO made in step S7 of FIG. 9 is equal to or more than a certain number of times after starting large intestine endoscopy based on the control information generated in step S200. Then, the processor 22 determines whether the actual insertion difficulty matches the insertion difficulty set in step S100. In the case of determination that the insertion difficulties match (YES in step S330), step S310 is performed again. On the other hand, in the case of determination that the insertion difficulties do not match (NO in step S330), the processor 22 performs processing of changing the condition (step S340) and then performs step S310 again. For example, in a case where the number of times of determination of NO made in step S2 of FIG. 9 is equal to or more than a certain number of times, the insertion portion 110 is not being inserted as planned. Therefore, the processor 22 makes determination of NO in step S330 because the actual insertion difficulty of the subject does not match the insertion difficulty set in step S100 of FIG. 10.

For example, in a case where the processor 22 makes determination of NO in step S2 because the predetermined loop is often formed, it is required the insertion portion 110 be inserted more carefully. Accordingly, for example, in a case where the initial operational difficulty set in step S100 is rank C, the processor 22 changes the operational difficulty from rank C to rank D in step S340. Then, the processor 22 generates control information based on rank D and performs automatic control of the endoscope 100 again on the basis of the control information.

Alternatively, for example, in a case where the rate of NO determined in step is lower than a certain rate, the processor 22 may make determination of NO in step S330 because the insertion portion 110 is being inserted too carefully. In this case, when the initial operational difficulty set in step S100 is rank C, the processor 22 changes the operational difficulty from rank C to rank B in step S340. Then, the processor 22 generates control information based on rank B and performs automatic control of the endoscope 100 again on the basis of the control information.

Consequently, in the information processing device 20 according to the present embodiment, the processor 22 acquires insertion situation information regarding the insertion situation of the endoscope 100, and changes, based on the acquired insertion situation information, the condition related to the generated control information (step S340). In this manner, it is possible to control the endoscope 100 on the basis of more appropriate control information after the endoscopy has been started.

For example, the control information generated in step S200 may also be control information regarding the operational manipulation of the endoscope 100. That is, in the information processing device 20 according to the present embodiment, the processor 22 generates control information regarding the operational manipulation of the endoscope 100 on the basis of the set condition. In this manner, it is possible to perform control of the endoscope 100 based on an appropriate operational manipulation depending on the attribute of the subject.

Although there have been proposed many operational manipulations for the endoscope 100 in large intestine endoscopy, the push method and the shaft retention shortening method are exemplified in the present embodiment. These two methods have a difference mainly in the insertion technique in the sigmoid colon and the like where the insertion difficulty is high as described above. Since the push method is a manipulation whose basic operation is to push the insertion portion 110, the predetermined loop described above may be formed. Thus, the push method is sometimes called loop method, loop formation method, and the like. For example, when a predetermined loop is formed to a large extent due to the insertion of the insertion portion 110 into the sigmoid colon by the push method, an operation of resolving the loop at an appropriate position on the further inner side than the SD junction is performed. The operation of resolving the predetermined loop is determined as appropriate depending on the type of the formed loop or the like. Note that adopting the push method not always results in formation of the predetermined loop.

The shaft retention shortening method is a manipulation that allows insertion from the sigmoid colon to the descending colon without forming the predetermined loop while avoiding such a strong pushing operation to place an excessive load on the intestinal tract and keeping the endoscope and the intestinal tract as straight as possible. The shaft retention shortening method may also called a straight method. In the present embodiment, control for insertion of the endoscope 100 to proceed is performed mainly by a technique that uses, for example, a hooking-the-fold method, and makes insertion to proceed while carefully hooking and flipping the folds of the large intestine one by one so as to pass over the folds.

The push method may also be subdivided into a plurality of types to constitute the control information. For example, the push method is classified into a “standard push method” and a “careful push method” in the present embodiment. The “standard push method” basically performs a pushing operation, and when a predetermined loop is formed, determination is made as to whether the insertion portion 110 can advance despite the formation of the loop, and when the insertion portion 110 can advance, the loop is not resolved. In a case where a technique by the “standard push method” is incorporated in the flowchart of FIG. 9, for example, the processor 22 makes determination of YES in step S2 of FIG. 9 even when a predetermined loop is formed. When determination of YES is made in the subsequent step S7, the processor 22 repeats steps S4 to S7. This allows large intestine endoscopy to be performed in a short time. The “careful push method” is common to the “standard push method” in basically performing the pushing operation, but differs from the “standard push method” in preventing formation of the predetermined loop at an early stage. For example, in a case where the “careful push method” is adopted, the standard value of similarity with the predetermined loop for which the determination of NO is made in step S2 of FIG. 9 is set lower than the standard value of a case of adopting the “standard push method”. That is, even with the low similarity between the shape of the insertion portion 110 when the endoscope 100 is being inserted and the shape of the predetermined loop, the processor 22 makes determination of NO in step S2 and executes the control program to resolve the loop in step S3. The operation amount per one operation and the operation speed in this case may be determined depending on the condition set based on the attribute of the subject described above. As described later, since the “careful push method” is a manipulation to be performed for subjects having a higher difficulty rank, the operation amount in the “careful push method” is generally smaller than the operation amount in the “standard push method”. Similarly, the operation speed in the “careful push method” is generally slower than the operation speed in the “standard push method”. Since the push method is a manipulation applicable to all subjects, the “careful push method” is ranked as the safest and most reliable manipulation.

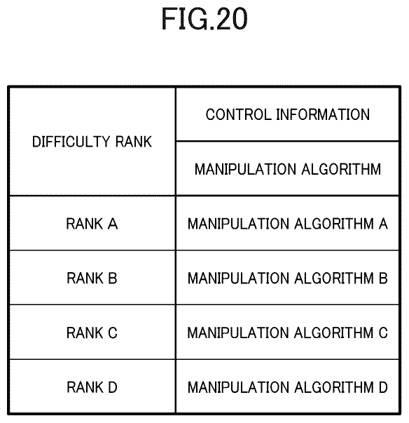

Various circumstances can be taken into consideration in determining whether to adopt the push method or the shaft retention shortening method for the subject. However, for example, one of the manipulations may be used to start large intestine endoscopy and then changed to the other one of the manipulations. That is, in the present embodiment, a manipulation algorithm that allows a plurality of manipulations to be combined may be constructed, and can be selected in step S200 depending on insertion difficulty. For example, as shown in the table of FIG. 20, in a case where the operational difficulty of the subject is set to rank A by step S100, a manipulation algorithm A is generated based on the table of FIG. 20 by step S200. Similarly, a manipulation algorithm B is generated by step S200 in a case where the operational difficulty of the subject is set to rank B by step S100, a manipulation algorithm C is generated by step S200 in a case where the operational difficulty of the subject is set to rank C by step S100, and a manipulation algorithm D is generated by step S200 in a case where the operational difficulty of the subject is set to rank D by step S100.

According to the manipulation algorithm A, the endoscope 100 is automatically controlled based on the “standard push method” at the start of large intestine endoscopy. According to the manipulation algorithm B, the endoscope 100 is automatically controlled based on the shaft retention shortening method at the start of large intestine endoscopy. According to the manipulation algorithm C, the endoscope 100 is automatically controlled based on the shaft retention shortening method at the start of large intestine endoscopy. According to the manipulation algorithm D, the endoscope 100 is automatically controlled based on the “careful push method” at the start of large intestine endoscopy. In this manner, in the information processing device 20 according to the present embodiment, the processor 22 generates control information in which the operational manipulation varies depending on the set condition. In this manner, it is possible to control the endoscope 100 on the basis of an appropriate operational manipulation depending on the attribute of the subject and the like.

The techniques described above with reference to FIG. 19 may be further combined. That is, the manipulation algorithms in FIG. 20 may be constructed to include algorithms for a plurality of operational manipulations and to allow the operational manipulation to be changed in the middle of the operation.

For example, the manipulation algorithm A includes an automatic control algorithm of the “standard push method” and an automatic control algorithm of the “careful push method”. Then, large intestine endoscopy is started by the “standard push method”. Subsequently, in a case where determination of NO is made in step S330 of FIG. 19, the processor 22 performs processing of changing the method to the “careful push method” as step S340.

For example, the manipulation algorithm B includes the automatic control algorithm of the “standard push method”, the automatic control algorithm of the “careful push method”, and an automatic control algorithm of the shaft retention shortening method. Then, large intestine endoscopy is started by the shaft retention shortening method. Subsequently, in a case where determination of NO is made in step S330 of FIG. 19, the processor 22 performs processing of changing the method to the “standard push method” as step S340. The shaft retention shortening method is said to be a less painful manipulation compared with the push method. However, in a predetermined case, the shaft retention shortening method is not applicable. Examples of the predetermined case are a case where adhesion of the sigmoid colon is found after large intestine endoscopy is started, a case where the sigmoid colon is folded in a manner more complicated than expected, and a case where the sigmoid colon is relaxed more than expected. In such cases, it is impossible to hook the folds of the large intestine and the insertion portion 110 does not advance as expected. Therefore, the processor 22 makes determination of NO in step S7 of FIG. 9 and further makes determination of NO in step S330 of FIG. 19. In a case where such a problem occurs as the subject complains of pain when the large intestine endoscopy is continued after the method is changed to the “standard push method”, the processor 22 makes determination of NO in step S2 of FIG. 10, and further makes determination of NO in step S330 of FIG. 19. As a result, the processor 22 performs processing of changing the operational manipulation from the “standard push method” to the “careful push method” as step S340.

For example, the manipulation algorithm C includes the automatic control algorithm of the “careful push method” and the automatic control algorithm of the shaft retention shortening method. Then, similarly to the manipulation algorithm B, large intestine endoscopy is started by the shaft retention shortening method. Subsequently, however, in a case where determination of NO is made in step S330 of FIG. 19, the processor 22 performs processing of changing the method to the “careful push method” as step S340. The subject to which the manipulation algorithm C is applied has higher operational difficulty than the subject to which the manipulation algorithm B is applied. Therefore, in a case where the shaft retention shortening method is not applicable, the method is immediately changed to the “careful push method”.

For example, the manipulation algorithm D includes only the automatic control algorithm of the “careful push method”. That is, the endoscope 100 is automatically controlled based on the “careful push method” at the start of large intestine endoscopy as described above, and the operational manipulation is not changed. This is because subjects with rank D having the highest operational difficulty often fall under the above-described predetermined cases where the shaft retention shortening method is not applicable.

For example, the properties of the endoscope 100 used may be further taken into consideration. For example, the push method is suitable for the endoscope 100 having the thin and soft insertion portion 110, while the shaft retention shortening method is suitable for the endoscope 100 having the thick and stiff insertion portion 110. Accordingly, for example, in a case where the gender of the subject as an attribute is female and the subject wishes to use the endoscope 100 having the thin and soft insertion portion 110, the processor 22 may set rank D as a condition in step S100 and perform processing of determining the manipulation algorithm D described above in step S200. For example, in a case where the processor 22 specifies the above-described manipulation algorithm B or manipulation algorithm C in step S200, both of the push method and the shaft retention shortening method can be selected. Therefore, the user may select the endoscope 100 including the insertion portion 110 to which both of the push method and the shaft retention shortening method is applicable.

Although FIG. 13 and the like show examples in which the condition setting (step S100) is performed by, for example, referring to the table, step S100 may also be performed by a technique of, for example, calculating information regarding operational difficulty.

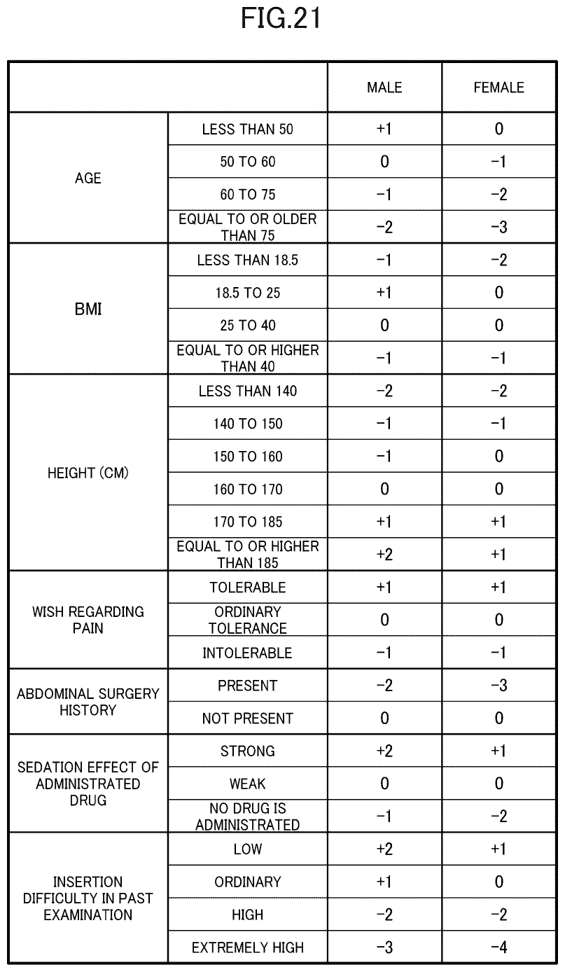

The information regarding operational difficulty is, for example, a number to quantify the operational difficulty. In the present embodiment, the number is referred to as an evaluation value. FIG. 21 shows examples of the evaluation value for each of the items. For example, in the case of males, the evaluation value is set to +1 for the age of less than 50, the evaluation value is set to 0 for the age of equal to or older than 50 and less than 60, the evaluation value is set to −1 for the age of equal to or older than 60 and less than 75, and the evaluation value is set to −2 for the age of equal to or older than 75. Similarly, for example, in the case of females, the evaluation value is set to 0 for the age of less than 50, the evaluation value is set to −1 for the age of equal to or older than 50 and less than 60, the evaluation value is set to −2 for the age of equal to or older than 60 and less than 75, and the evaluation value is set to −3 for the age of equal to or older than 75.

For example, in the case of males, the evaluation value is set to −1 for the BMI of less than 18.5, the evaluation value is set to +1 for the BMI of equal to or higher than 18.5 and less than 25, the evaluation value is set to 0 for the BMI of equal to or higher than 25 and less than 40, and the evaluation value is set to −1 for the BMI of equal to or higher than 40. Similarly, for example, in the case of females, the evaluation value is set to −2 for the BMI of less than 18.5, the evaluation value is set to 0 for the BMI of equal to or higher than 18.5 and less than 25, the evaluation value is set to 0 for the BMI of equal to or higher than 25 and less than 40, and the evaluation value is set to −1 for the BMI of equal to or higher than 40.

For example, in the case of males, the evaluation value is set to −2 for the height of less than 140 cm, the evaluation value is set to −1 for the height of equal to or higher than 140 cm and less than 150 cm, the evaluation value is set to −1 for the height of equal to or higher than 150 cm and less than 160 cm, and the evaluation value is set to 0 for the height of equal to or higher than 160 cm and less than 170 cm. For example, in the case of males, the evaluation value is set to +1 for the height of equal to or higher than 170 cm and less than 185 cm, and the evaluation value is set to +2 for the height of equal to or higher than 185 cm. Similarly, for example, in the case of females, the evaluation value is set to −2 for the height of less than 140 cm, the evaluation value is set to −1 for the height of equal to or higher than 140 cm and less than 150 cm, the evaluation value is set to 0 for the height of equal to or higher than 150 cm and less than 160 cm, and the evaluation value is set to 0 for the height of equal to or higher than 160 cm and less than 170 cm. For example, in the case of females, the evaluation value is set to +1 for the height of equal to or higher than 170 cm and less than 185 cm, and the evaluation value is set to +1 for the height of equal to or higher than 185 cm.

For example, as a result of an interview with a male subject about his wish regarding pain, the evaluation value is set to +1 for an answer that the subject can tolerate pain, the evaluation value is set to 0 for an answer that the subject has moderate tolerance to pain, and the evaluation value is set to −1 for an answer that the subject cannot tolerate pain. Similarly, for example, as a result of an interview with a female subject about her wish regarding pain, the evaluation value is set to +1 for an answer that the subject can tolerate pain, the evaluation value is set to 0 for an answer that the subject has moderate tolerance to pain, and the evaluation value is set to −1 for an answer that the subject cannot tolerate pain.

For example, in the case of male subjects, the evaluation value is set to −2 for a subject with a history of abdominal surgery, and the evaluation value is set to 0 for a subject without a history of abdominal surgery. Similarly, for example, in the case of female subjects, the evaluation value is set to −3 for a subject with a history of abdominal surgery, and the evaluation value is set to 0 for a subject without a history of abdominal surgery.

For example, in a case where sedation is combined with endoscopy for a male subject, the evaluation value is set to +2 when a sedation effect of a drug to be administrated is strong, and the evaluation value is set to 0 when a sedation effect of a drug to be administrated is weak. For example, the evaluation value is set to −1 when a drug having a sedation effect is not administrated to a male subject. Similarly, for example, in a case where sedation is combined with endoscopy for a female subject, the evaluation value is set to +1 when a sedation effect of a drug to be administrated is strong, and the evaluation value is set to 0 when a sedation effect of a drug to be administrated is weak. For example, the evaluation value is set to −2 when a drug having a sedation effect is not administrated to a female subject.

For example, for a male subject, the evaluation value is set to +2 in a case where the record of a past examination shows low insertion difficulty, and the evaluation value is set to +1 in a case where the record of a past examination shows ordinary insertion difficulty. For example, for a male subject, the evaluation value is set to −2 in a case where the record of a past examination shows high insertion difficulty, and the evaluation value is set to −3 in a case where the record of a past examination shows extremely high insertion difficulty. Similarly, for example, for a female subject, the evaluation value is set to +1 in a case where the record of a past examination shows low insertion difficulty, and the evaluation value is set to 0 in a case where the record of a past examination shows ordinary insertion difficulty. For example, for a female subject, the evaluation value is set to −2 in a case where the record of a past examination shows high insertion difficulty, and the evaluation value is set to −4 in a case where the record of a past examination shows extremely high insertion difficulty.

Then, in step S100, the processor 22 calculates the sum of the evaluation values of the respective items in FIG. 21. For example, in a case where the gender is male, the range of the possible evaluation value is within −12 to +9. Similarly, in a case where the gender is female, the range of the possible evaluation value is within −17 to +4. Note that using the evaluation values of all items in FIG. 21 is not required, and the user can select the evaluation values to use as appropriate. For example, it is supposed the user selects gender, age, BMI, and height as attributes. In a case where the gender of a subject is male, the age of the subject is 36, the BMI of the subject is 22, and the height of the subject is 175 cm, the sum of the evaluation values is +3 with reference to FIG. 21. Similarly, in a case where the gender of a subject is female, the age of the subject is 70, the BMI of the subject is 18, and the height of the subject is 145 cm, the sum of the evaluation values is-5 with reference to FIG. 21.

Then, the processor 22 sets the difficulty rank according to, for example, the sum of the obtained evaluation values. For example, in a case where the sum of the evaluation values for a subject is in the range of +1 to +9, the processor 22 sets the difficulty rank for the subject to rank A. For example, in a case where the sum of the evaluation values for a subject is in the range of −3 to 0, the processor 22 sets the difficulty rank for the subject to rank B. For example, in a case where the sum of the evaluation values for a subject is in the range of −7 to −4, the processor 22 sets the difficulty rank for the subject to rank C. For example, in a case where the sum of the evaluation values for a subject is in the range of −17 to −8, the processor 22 sets the difficulty rank for the subject to rank D. Then, in step S200, the processor 22 generates control information in accordance with the set ranks A to D.

Note that the information regarding the operational difficulty, for example, is not limited to numbers, but may be symbols such as alphabets, stars, or the like. Such symbols may be included as a part of the table of FIG. 21 to calculate the insertion difficulty. For example, in consideration of a predetermined past result, the field corresponding to a female subject with the age of equal to or older than 75 is input with an alphabet letter “D” instead of a number. Then, in step S100, the difficulty rank may be set to rank D in a case where the calculation result based on the table of FIG. 21 includes “D”. An example of the predetermined past result is that a subject who is old and female often results in the difficulty rank of rank D regardless of whether to take other items into consideration. Similarly, the same information as “D” described above may be assigned to, for example, an item corresponding to a case with a history of abdominal surgery or an item corresponding to a case of extremely high insertion difficulty in the past examination.

Consequently, in the information processing device 20 according to the present embodiment, the processor 22 calculates information regarding the difficulty of insertion of the endoscope 100, classifies the calculated information into a predetermined rank, and generates control information on the basis of the classified predetermined rank. In this manner, it is possible to evaluate the insertion difficulty of the endoscope 100 by calculation. For example, when many matters are to be considered for the insertion difficulty, enabling quantification of the insertion difficulty for each item may be convenient.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. An information processing device comprising a processor including hardware,
wherein the processor is configured to:
acquire a physical attribute of a subject;
set, based on the acquired physical attribute of the subject, a condition related control information for insertion of an endoscope; and
generate, based on the set condition, the control information.

2. The information processing device according to claim 1,
wherein the processor generates, as the control information, an operation amount or an operation speed.

3. The information processing device according to claim 2,
wherein the processor
sets, based on the physical attribute of the subject, a rank of operational difficulty as the condition, and
generates, based on the set condition, the control information,
the operation amount is reduced by a predetermined amount as the operational difficulty increases, and
the operation speed is reduced to a predetermined speed as the operational difficulty increases.

4. The information processing device according to claim 3,
wherein the processor
generates, as the operation amount, a forward/backward movement operation amount related to forward/backward movement of the endoscope and a rotation operation amount related to rotation of the endoscope, and
generates, as the operation speed, a speed at which the endoscope moves forward and backward and a speed at which the endoscope rotates.

5. The information processing device according to claim 3,
wherein the processor performs control such that, in a case of determination that the operational difficulty is high, at least one of the operation amount and the operation speed as the control information is reduced compared with a case of determination that the operational difficulty is low.

6. The information processing device according to claim 3,
wherein the processor performs control such that, in a case of determination that the operational difficulty is high, both of the operation amount and the operation speed as the control information is reduced compared with a case of determination that the operational difficulty is low.

7. The information processing device according to claim 3,
wherein the processor
acquires, as the physical attribute, first physical attribute and second physical attribute,
determines the operational difficulty based on the first physical attribute and the second physical attribute, and
generates the control information based on the first physical attribute or the second physical attribute for which the operational difficulty has been determined to be highest.

8. The information processing device according to claim 7,
wherein the processor acquires, as the first physical attribute and the second physical attribute, at least two of the physical attributes among age, gender, height, weight, and BMI of the subject.

9. The information processing device according to claim 1,
wherein the processor
acquires a resistance force generated between an endoscope insertion portion and an intestinal tract, and
generates the control information based on the resistance force.

10. The information processing device according to claim 1,
wherein the processor generates the control information based on a position of distal end portion of an endoscope insertion portion.

11. The information processing device according to claim 10,
wherein the processor switches operation to allow a user to operate the endoscope when the distal end portion is in any of an ascending colon, a descending colon, and a rectum.

12. The information processing device according to claim 1,
wherein the processor
acquires examination information that is information regarding an examination, and
sets, based on the physical attribute of the subject and the examination information, the condition related to the control information.

13. The information processing device according to claim 12,
wherein the processor
acquires the examination information including sedation information that is information regarding sedation, and
sets, based on the physical attribute of the subject and the examination information, the condition related to the control information.

14. The information processing device according to claim 12,
wherein the processor performs control such that, in a case where a drug having a sedation effect is administered, at least one of the operation amount and the operation speed as the control information is increased compared with a case where the drug is not administered.

15. The information processing device according to claim 12,
wherein the processor performs control such that, in a case where a drug having a strong sedation effect is administered, at least one of the operation amount and the operation speed as the control information is increased compared with a case where a drug having a weak sedation effect is administered.

16. The information processing device according to claim 1,
wherein the processor
acquires insertion situation information regarding an insertion situation of the endoscope, and
changes, based on the acquired insertion situation information, the condition related to the generated control information.

17. The information processing device according to claim 1,
wherein the processor generates, based on the set condition, the control information regarding an operational manipulation of the endoscope.

18. The information processing device according to claim 17,
wherein the processor generates the control information in which the operational manipulation varies depending on the set condition.

19. An endoscopic system comprising:
an endoscope configured to be electrically driven;
a drive device configured to drive the endoscope; and
a processor including hardware,
wherein the processor is configured to:
acquire a physical attribute of a subject;
set, based on the acquired physical attribute of the subject, a condition related control information for insertion of the endoscope; and
generate, based on the set condition, the control information for controlling the drive device.

20. A method for controlling an information processing device,
wherein the information processing device is configured to:
acquire a physical attribute of a subject;
set, based on the acquired physical attribute of the subject, a condition related control information for insertion of an endoscope; and
generate, based on the set condition, the control information.
